(19) United States
(12) Patent Application Publication
(10) Pub. No.: US 2026/0051399 A1
(43) Pub. Date: Feb. 19, 2026

(54) SYSTEMS AND METHODS FOR PROVIDING A SLEEP THERAPY COMMUNITY

(71) Applicant: RESMED DIGITAL HEALTH INC., San Diego, CA (US)

(72) Inventors: Jamie Graeme WEHBEH, Bella Vista (AU); James Douglas WEBSTER, Bella Vista (AU); Sakeena DE SOUZA, Sydney (AU); Michael James DENT, Bella Vista (AU); Siew Ting OOI, Singapore (SG); Jaiden James CHOY, Bella Vista (AU); Priyanshu GUPTA, Sydney (AU); Garth Alan BERRIMAN, Bella Vista (AU); Andrew CHAN, Bella Vista (AU)

(21) Appl. No.: 19/101,941

(22) PCT Filed: Aug. 16, 2023
(86) PCT No.: PCT/US2023/030402

§ 371 Date: Feb. 7, 2025

Related U.S. Application Data
(60) Provisional application No. 63/371,921, filed on Aug. 19, 2022.

(51) Int. Cl. G16H 40/67; A61M 16/00; A61M 21/02

(52) U.S. Cl. CPC G16H 40/67; A61M 16/024; A61M 21/02; A61M 2205/3303; A61M 2205/3553; A61M 2205/3584; A61M 2205/502; A61M 2205/52

(57) ABSTRACT

A method includes storing, in a database, sleep information associated with a plurality of users, the plurality of users including a first subset of users having a first common feature in the sleep information. The method also includes defining, by a control system based on the first common feature, a first user group comprising the first subset of users. The method also includes receiving, via the control system from a user device, sleep information associated with a first user. The method also includes determining, by the control system based on the sleep information associated with the first user, whether the first user has the first common feature. The method also includes, responsive to determining that the first user has the first common feature, assigning, by the control system, the first user as a constituent of the first user group.

### CROSS-REFERENCE TO RELATED APPLICATIONS

This application claims the benefit of, and priority to, U.S. Provisional Ser. No. 63/371,921, filed Aug. 19, 2022, which is hereby incorporated by reference herein in its entirety.

### TECHNICAL FIELD

The present disclosure relates generally to systems and methods for matching a sleep therapy application user to a user community, and more particularly, to systems and methods for determining match between a user and a user group of a plurality of users based on sleep information of the user.

### BACKGROUND

Many individuals suffer from sleep-related and/or respiratory-related disorders such as, for example, Sleep Disordered Breathing (SDB), which can include Obstructive Sleep Apnea (OSA), Central Sleep Apnea (CSA), other types of apneas such as mixed apneas and hypopneas, Respiratory Effort Related Arousal (RERA), and snoring. In some cases, these disorders manifest, or manifest more pronouncedly, when the individual is in a particular lying/sleeping position. These individuals may also suffer from other health conditions (which may be referred to as comorbidities), such as insomnia (e.g., difficulty initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders.

These disorders are often treated using a respiratory therapy system (e.g., a continuous positive airway pressure (CPAP) system), which delivers pressurized air to aid in preventing the individual's airway from narrowing or collapsing during sleep. However, some users find such systems to be uncomfortable, difficult to use, expensive, aesthetically unappealing and/or fail to perceive the benefits associated with using the system. As a result, some users will elect not to use the respiratory therapy system or discontinue use of the respiratory therapy system absent a demonstration of the severity of their symptoms when respiratory therapy treatment is not used or encouragement or affirmation that the respiratory therapy system is improving their sleep quality and reducing the symptoms of these disorders. The present disclosure is directed to solving these and other problems.

### SUMMARY

According to some implementations of the present disclosure, a method includes storing, in a database, sleep information associated with a plurality of users, the plurality of users including a first subset of users having a first common feature in the sleep information. The method also includes defining, by a control system based on the first common feature, a first user group comprising the first subset of users. The method also includes receiving, via the control system from a user device, sleep information associated with a first user. The method also includes determining, by the control system based on the sleep information associated with the first user, whether the first user has the first common feature. The method also includes responsive to determining that the first user has the first common feature, assigning, by the control system, the first user as a constituent of the first user group.

According to some implementations of the present disclosure, a system includes a database, wherein the database is configured to store sleep information associated with a plurality of users. The system also includes a control system communicatively coupled to the database, wherein the control system is configured to define, based on a first common feature, a first user group comprising a first subset of users of the plurality of users, the first subset of users having the first common feature in the sleep information. The control system is further configured to receive, from a first user device, sleep information associated with a first user. The control system is further configured to determine, based on the sleep information associated with the first user, whether the first user has the first common feature. The control system is further configured to responsive to determining that the first user has the first common feature, assigning the first user as constituent of the first user group.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2 is a perspective view of at least a portion of the system of FIG. 1, a user, and a bed partner, according to some implementations of the present disclosure;

FIG. 3A is a perspective view of a respiratory therapy device of the system of FIG. 1, according to some implementations of the present disclosure;

FIG. 3B is a perspective view of the respiratory therapy device of FIG. 3A illustrating an interior of a housing, according to some implementations of the present disclosure;

FIG. 4A is a perspective view of a user interface, according to some implementations of the present disclosure;

FIG. 4B is an exploded view of the user interface of FIG. 4A, according to some implementations of the present disclosure;

FIG. 5A is a perspective view of a user interface, according to some implementations of the present disclosure;

FIG. 5B is an exploded view of the user interface of FIG. 5A, according to some implementations of the present disclosure;

FIG. 6A is a perspective view of a user interface, according to some implementations of the present disclosure;

FIG. 6B is an exploded view of the user interface of FIG. 6A, according to some implementations of the present disclosure;

FIG. 7 illustrates an exemplary timeline for a sleep session, according to some implementations of the present disclosure;

FIG. 8 illustrates an exemplary hypnogram associated with the sleep session of FIG. 7, according to some implementations of the present disclosure;

FIGS. 9A and 9B depict a user device presenting a user's personal homepage, including the user's sleep information and personal information, according to some implementation of the present disclosure;

FIGS. 10A and 10B depict a user device presenting a landing page of a user group of which the user is a member, according to some implementations of the present disclosure;

FIG. 11A-11C depict a user device presenting recommendations based on the user's sleep information and/or personal information, according to some implementations of the present disclosure;

FIG. 12A-12C depict a user device presenting participatory challenges in which the first user 1206 can participate, according to some implementations of the present disclosure;

FIG. 13 is a block diagram of a system for assigning users to groups based on sleep information, according to some implementations of the present disclosure; and

FIG. 14 is a flow chart depicting example operations for assigning users to groups based on sleep information, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory disorders, such as Sleep Disordered Breathing (SDB) such as Obstructive Sleep Apnea (OSA), Central Sleep Apnea (CSA) and other types of apneas, Respiratory Effort Related Arousal (RERA), snoring, Cheyne-Stokes Respiration (CSR), respiratory insufficiency, Obesity Hyperventilation Syndrome (OHS), Chronic Obstructive Pulmonary Disease (COPD), Periodic Limb Movement Disorder (PLMD), Restless Leg Syndrome (RLS), Neuromuscular Disease (NMD), and chest wall disorders.

Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). CSA results when the brain temporarily stops sending signals to the muscles that control breathing. Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events fulfil the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung. COPD encompasses a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

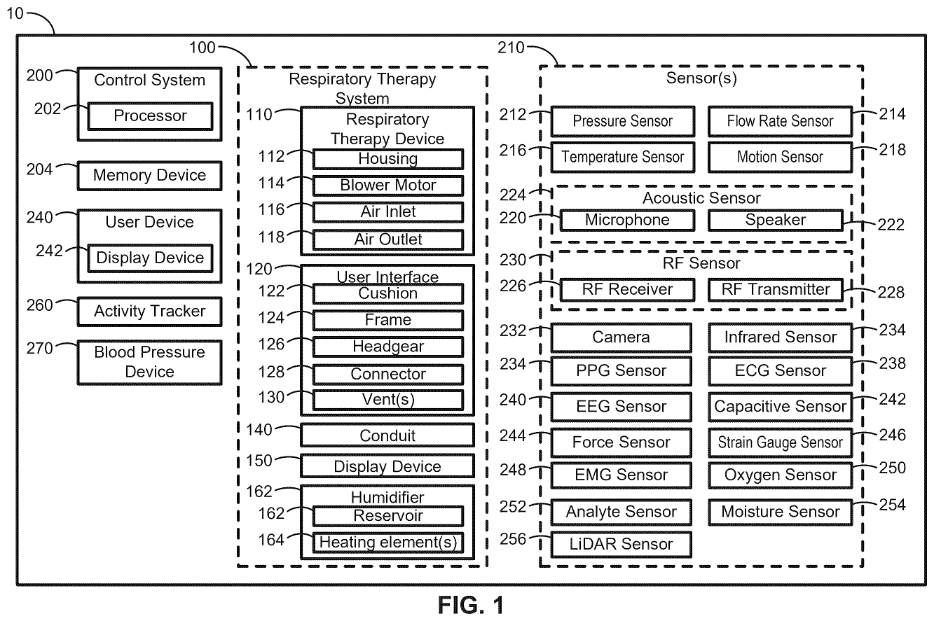

Referring to FIG. 1, a system 10, according to some implementations of the present disclosure, is illustrated. The system 10 includes a respiratory therapy system 100, a control system 200, one or more sensors 210, a user device 260, and an activity tracker 270.

The respiratory therapy system 100 includes a respiratory pressure therapy (RPT) device 110 (referred to herein as respiratory therapy device 110), a user interface 120 (also referred to as a mask or a patient interface), a conduit 140 (also referred to as a tube or an air circuit), a display device 150, and a humidifier 160. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 100 is generally used to treat individuals suffering from one or more sleep-related respiratory disorders (e.g., obstructive sleep apnea, central sleep apnea, or mixed sleep apnea).

The respiratory therapy system 100 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

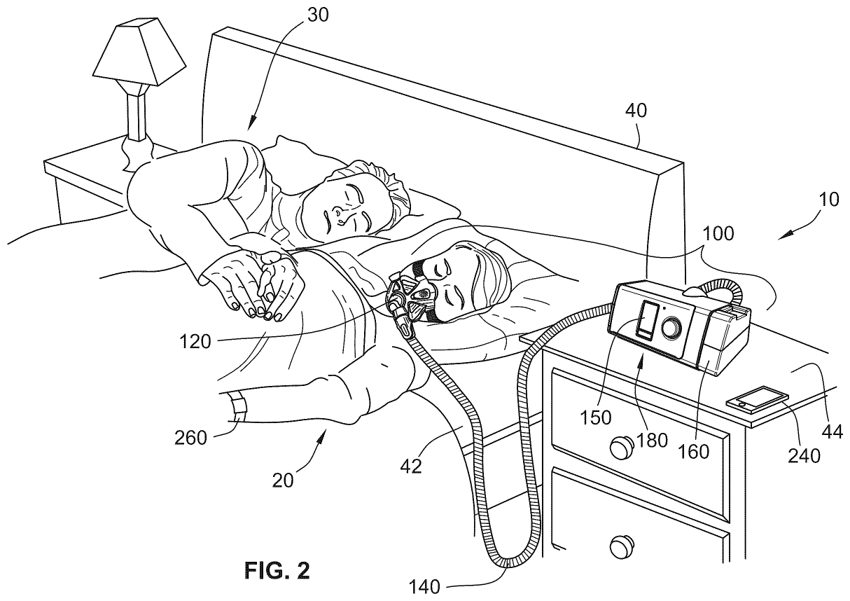

As shown in FIG. 2, the respiratory therapy system 100 can be used to treat user 20. In this example, the user 20 of the respiratory therapy system 100 and a bed partner 30 are located in a bed 40 and are laying on a mattress 42. The user interface 120 can be worn by the user 20 during a sleep session. The respiratory therapy system 100 generally aids in increasing the air pressure in the throat of the user 20 to aid in preventing the airway from closing and/or narrowing during sleep. The respiratory therapy device 110 can be positioned on a nightstand 44 that is directly adjacent to the bed 40 as shown in FIG. 2, or more generally, on any surface or structure that is generally adjacent to the bed 40 and/or the user 20.

The respiratory therapy device 110 is generally used to generate pressurized air that is delivered to a user (e.g., using one or more motors that drive one or more compressors). In some implementations, the respiratory therapy device 110 generates continuous constant air pressure that is delivered to the user. In other implementations, the respiratory therapy device 110 generates two or more predetermined pressures (e.g., a first predetermined air pressure and a second predetermined air pressure). In still other implementations, the respiratory therapy device 110 generates a variety of different air pressures within a predetermined range. For example, the respiratory therapy device 110 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory therapy device 110 can also deliver pressurized air at a predetermined flow rate between, for example, about −20 L/min and about 150 L/min, while maintaining a positive pressure (relative to the ambient pressure).

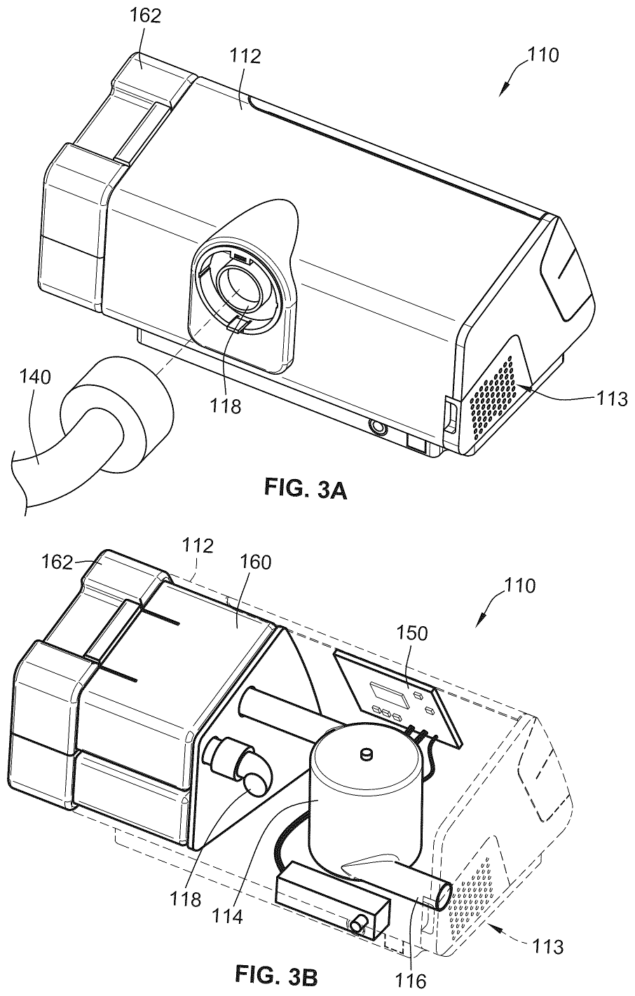

The respiratory therapy device 110 includes a housing 112, a blower motor 114, an air inlet 116, and an air outlet 118 (FIG. 1). Referring to FIGS. 3A and 3B, the blower motor 114 is at least partially disposed or integrated within the housing 112. The blower motor 114 draws air from outside the housing 112 (e.g., atmosphere) via the air inlet 116 and causes pressurized air to flow through the humidifier 160, and through the air outlet 118. In some implementations, the air inlet 116 and/or the air outlet 118 include a cover that is moveable between a closed position and an open position (e.g., to prevent or inhibit air from flowing through the air inlet 116 or the air outlet 118). As shown in FIGS. 3A and 3B, the housing 112 can include a vent 113 to allow air to pass through the housing 112 to the air inlet 116. As described below, the conduit 140 is coupled to the air outlet 118 of the respiratory therapy device 110.

Referring back to FIG. 1, the user interface 120 engages a portion of the user's face and delivers pressurized air from the respiratory therapy device 110 to the user's airway to aid in preventing the airway from narrowing and/or collapsing during sleep. This may also increase the user's oxygen intake during sleep. Generally, the user interface 120 engages the user's face such that the pressurized air is delivered to the user's airway via the user's mouth, the user's nose, or both the user's mouth and nose. Together, the respiratory therapy device 110, the user interface 120, and the conduit 140 form an air pathway fluidly coupled with an airway of the user. The pressurized air also increases the user's oxygen intake during sleep. Depending upon the therapy to be applied, the user interface 120 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cmH₂O.

The user interface 120 can include, for example, a cushion 122, a frame 124, a headgear 126, connector 128, and one or more vents 130. The cushion 122 and the frame 124 define a volume of space around the mouth and/or nose of the user. When the respiratory therapy system 100 is in use, this volume space receives pressurized air (e.g., from the respiratory therapy device 110 via the conduit 140) for passage into the airway(s) of the user. The headgear 126 is generally used to aid in positioning and/or stabilizing the user interface 120 on a portion of the user (e.g., the face), and along with the cushion 122 (which, for example, can comprise silicone, plastic, foam, etc.) aids in providing a substantially air-tight seal between the user interface 120 and the user 20. In some implementations the headgear 126 includes one or more straps (e.g., including hook and loop fasteners). The connector 128 is generally used to couple (e.g., connect and fluidly couple) the conduit 140 to the cushion 122 and/or frame 124. Alternatively, the conduit 140 can be directly coupled to the cushion 122 and/or frame 124 without the connector 128. The vent 130 can be used for permitting the escape of carbon dioxide and other gases exhaled by the user 20. The user interface 120 generally can include any suitable number of vents (e.g., one, two, five, ten, etc.).

As shown in FIG. 2, in some implementations, the user interface 120 is a facial mask (e.g., a full face mask) that covers at least a portion of the nose and mouth of the user 20. Alternatively, the user interface 120 can be a nasal mask that provides air to the nose of the user or a nasal pillow mask that delivers air directly to the nostrils of the user 20. In other implementations, the user interface 120 includes a mouthpiece (e.g., a night guard mouthpiece molded to conform to the teeth of the user, a mandibular repositioning device, etc.).

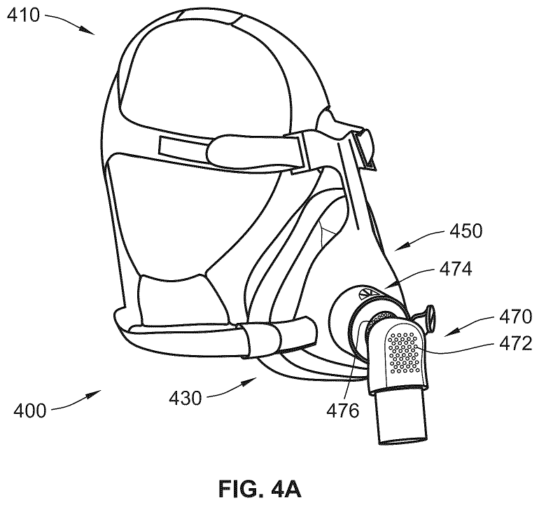

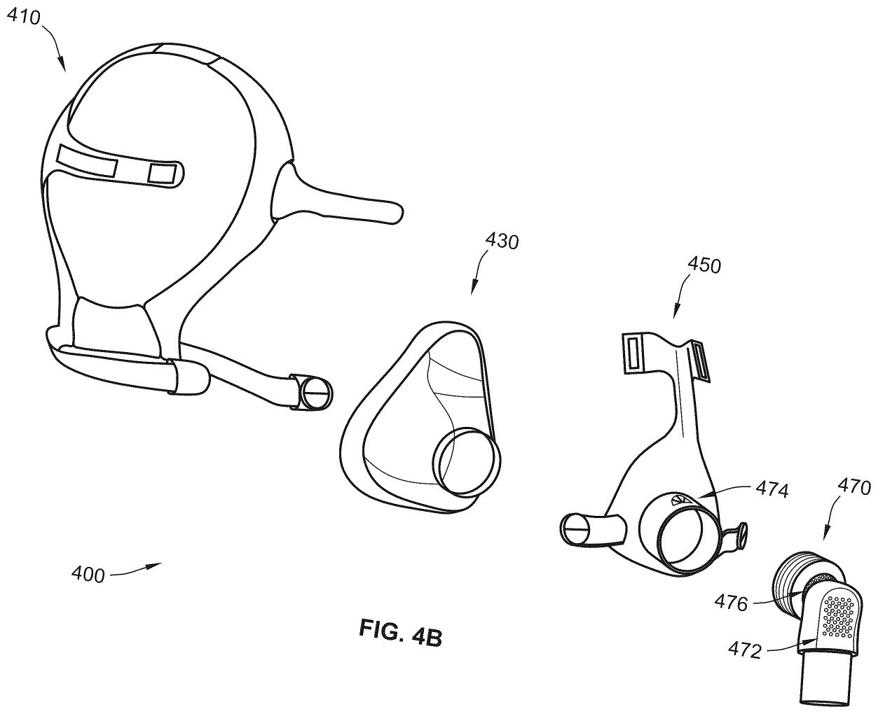

Referring to FIGS. 4A and 4B, a user interface 400 that is the same as, or similar to, the user interface 120 (FIG. 1) according to some implementations of the present disclosure is illustrated. The user interface 400 generally includes a cushion 430 and a frame 450 that define a volume of space around the mouth and/or nose of the user. When in use, the volume of space receives pressurized air for passage into the user's airways. In some implementations, the cushion 430 and frame 450 of the user interface 400 form a unitary component of the user interface. The user interface 400 can also include a headgear 410, which generally includes a strap assembly and optionally a connector 470. The headgear 410 is configured to be positioned generally about at least a portion of a user's head when the user wears the user interface 400. The headgear 410 can be coupled to the frame 450 and positioned on the user's head such that the user's head is positioned between the headgear 410 and the frame 450. The cushion 430 is positioned between the user's face and the frame 450 to form a seal on the user's face. The optional connector 470 is configured to couple to the frame 450 and/or cushion 430 at one end and to a conduit of a respiratory therapy device (not shown). The pressurized air can flow directly from the conduit of the respiratory therapy system into the volume of space defined by the cushion 430 (or cushion 430 and frame 450) of the user interface 400 through the connector 470). From the user interface 400, the pressurized air reaches the user's airway through the user's mouth, nose, or both. Alternatively, where the user interface 400 does not include the connector 470, the conduit of the respiratory therapy system can connect directly to the cushion 430 and/or the frame 450.

In some implementations, the connector 470 may include one or more vents 472 (e.g., a plurality of vents) located on the main body of the connector 470 itself and/or one or a plurality of vents 476 (“diffuser vents”) in proximity to the frame 450, for permitting the escape of carbon dioxide (CO₂) and other gases exhaled by the user. In some implementations, one or a plurality of vents, such as vents 472 and/or 476 may be located in the user interface 400, such as in frame 450, and/or in the conduit 140. In some implementations, the frame 450 includes at least one anti-asphyxia valve (AAV) 474, which allows CO₂ and other gases exhaled by the user to escape in the event that the vents (e.g., the vents 472 or 476) fail when the respiratory therapy device is active. In general, AAVs (e.g., the AAV 474) are present for full face masks (e.g., as a safety feature); however, the diffuser vents and vents located on the mask or connector (usually an array of orifices in the mask material itself or a mesh made of some sort of fabric, in many cases replaceable) are not necessarily both present (e.g., some masks might have only the diffuser vents such as the plurality of vents 476, other masks might have only the plurality of vents 472 on the connector itself).

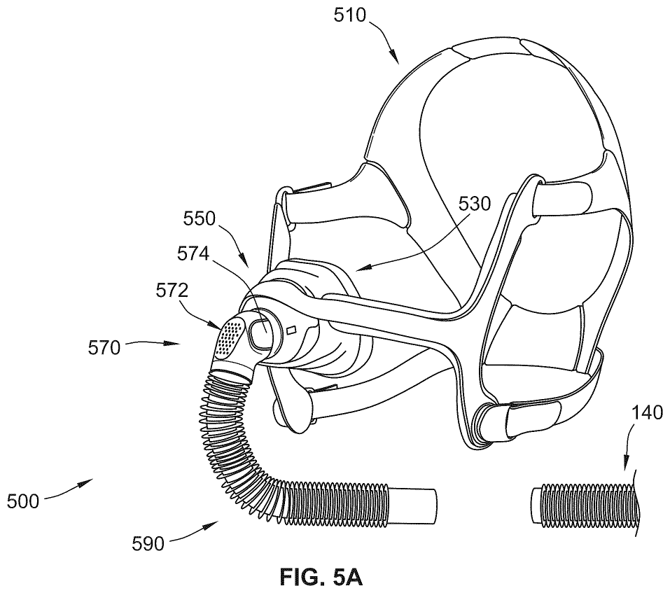

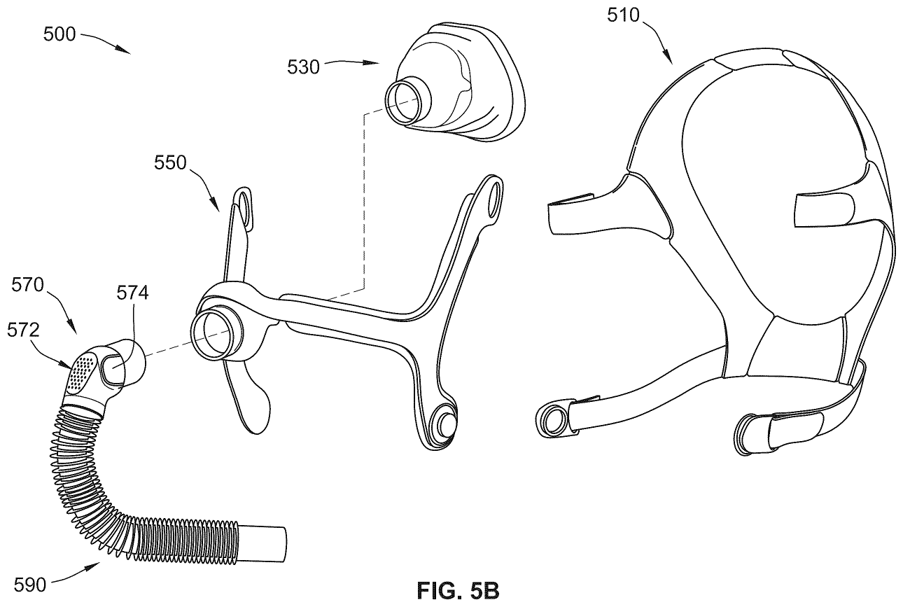

Referring to FIGS. 5A and 5B, a user interface 500 that the is the same, or similar to, the user interface 120 (FIG. 1) according to some implementations of the present disclosure is illustrated. The user interface 500 differs from the user interface 400 (FIGS. 4A and 4B) in that the user interface 500 is an indirect user interface, whereas the user interface 400 is a direct user interface. The interface 500 includes a headgear 510 (e.g., as a strap assembly), a cushion 530, a frame 550, a connector 570, and a user interface conduit 590 (often referred to as a minitube or a flexitube). The user interface 500 is an indirectly connected user interface because pressurized air is delivered from the conduit 140 of the respiratory therapy system to the cushion 530 and/or frame 550 through the user interface conduit 590, rather than directly from the conduit 140 of the respiratory therapy system.

In some implementations, the cushion 530 and frame 550 form a unitary component of the user interface 500. Generally, the user interface conduit 590 is more flexible than the conduit 140 of the respiratory therapy system 100 (FIG. 1) described above and/or has a diameter smaller than the diameter of the than the than the conduit 140. The user interface conduit 590 is typically shorter that conduit 140. Similar to the headgear 310 of user interface 300 (FIGS. 3A-3B), the headgear 510 of user interface 500 is configured to be positioned generally about at least a portion of a user's head when the user wears the user interface 500. The headgear 510 can be coupled to the frame 550 and positioned on the user's head such that the user's head is positioned between the headgear 510 and the frame 550. The cushion 530 is positioned between the user's face and the frame 550 to form a seal on the user's face. The connector 570 is configured to couple to the frame 550 and/or cushion 530 at one end and to the conduit 590 of the user interface 500 at the other end. In other implementations, the conduit 590 may connect directly to frame 550 and/or cushion 530. The conduit 590, at the opposite end relative to the frame 550 and cushion 530, is configured to connect to the conduit 140. The pressurized air can flow from the conduit 140 of the respiratory therapy system, through the user interface conduit 590, and the connector 570, and into a volume of space define by the cushion 530 (or cushion 530 and frame 550) of the user interface 500 against a user's face. From the volume of space, the pressurized air reaches the user's airway through the user's mouth, nose, or both.

In some implementations, the connector 570 includes a plurality of vents 572 for permitting the escape of carbon dioxide (CO₂) and other gases exhaled by the user when the respiratory therapy device is active. In such implementations, each of the plurality of vents 572 is an opening that may be angled relative to the thickness of the connector wall through which the opening is formed. The angled openings can reduce noise of the CO₂ and other gases escaping to the atmosphere. Because of the reduced noise, acoustic signal associated with the plurality of vents 572 may be more apparent to an internal microphone, as opposed to an external microphone. Thus, an internal microphone may be located within, or otherwise physically integrated with, the respiratory therapy system and in acoustic communication with the flow of air which, in operation, is generated by the flow generator of the respiratory therapy device, and passes through the conduit and to the user interface 500.

In some implementations, the connector 570 optionally includes at least one valve 574 for permitting the escape of CO₂ and other gases exhaled by the user when the respiratory therapy device is inactive. In some implementations, the valve 574 (an example of an anti-asphyxia valve) includes a silicone (or other suitable material) flap that is a failsafe component, which allows CO₂ and other gases exhaled by the user to escape in the event that the vents 572 fail when the respiratory therapy device is active. In such implementations, when the silicone flap is open, the valve opening is much greater than each vent opening, and therefore less likely to be blocked by occlusion materials.

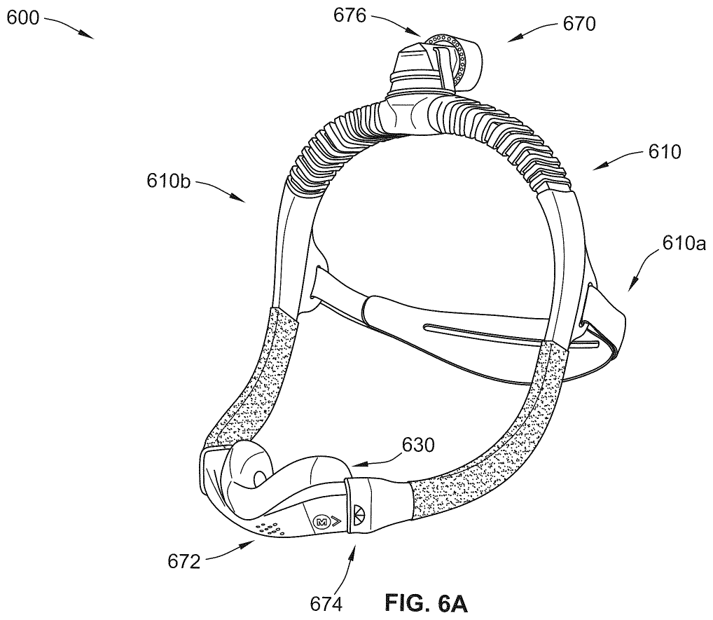

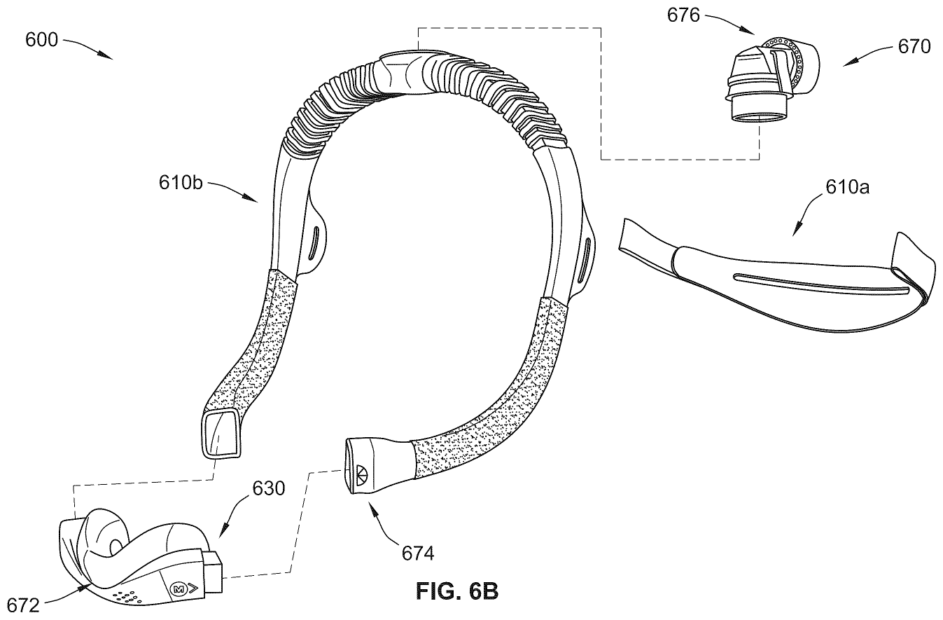

Referring to FIGS. 6A and 6B, a user interface 600 that is the same as, or similar to, the user interface 120 (FIG. 1) according to some implementations of the present disclosure is illustrated. The user interface 600 is similar to the user interface 500 in that it is an indirect user interface. The indirect headgear user interface 600 includes headgear 610, a cushion 630, and a connector 670. The headgear 610 includes strap 610a and a headgear conduit 610b. Similar to the user interface 400 (FIGS. 4A-4B) and user interface 500 (FIGS. 5A-5B), the headgear 610 is configured to be positioned generally about at least a portion of a user's head when the user wears the user interface 600. The headgear 610 includes a strap 610a that can be coupled to the headgear conduit 610b and positioned on the user's head such that the user's head is positioned between the strap 610a and the headgear conduit 610b. The cushion 630 is positioned between the user's face and the headgear conduit 610b to form a seal on the user's face.

The connector 670 is configured to couple to the headgear 610 at one end and a conduit of the respiratory therapy system at the other end (e.g., conduit 140). In other implementations, the connector 670 is not included and the headgear 610 can alternatively connect directly to conduit of the respiratory therapy system. The headgear conduit 610b can be configured to deliver pressurized air from the conduit of the respiratory therapy system to the cushion 630, or more specifically, to the volume of space around the mouth and/or nose of the user and enclosed by the user cushion. The headgear conduit 610b is hollow to provide a passageway for the pressurized air. Both sides of the headgear conduit 610b can be hollow to provide two passageways for the pressurized air. Alternatively, only one side of the headgear conduit 610b can be hollow to provide a single passageway. In the implementation illustrated in FIGS. 6A and 6B, headgear conduit 610b comprises two passageways which, in use, are positioned at either side of a user's head/face. Alternatively, only one passageway of the headgear conduit 610b can be hollow to provide a single passageway. The pressurized air can flow from the conduit of the respiratory therapy system, through the connector 670 and the headgear conduit 610b, and into the volume of space between the cushion 630 and the user's face. From the volume of space between the cushion 630 and the user's face, the pressurized air reaches the user's airway through the user's mouth, nose, or both.

In some implementations, the cushion 630 includes a plurality of vents 672 on the cushion 630 itself. Additionally, or alternatively, in some implementations, the connector 670 includes a plurality of vents 676 (“diffuser vents”) in proximity to the headgear 610, for permitting the escape of carbon dioxide (CO₂) and other gases exhaled by the user when the respiratory therapy device is active. In some implementations, the headgear 610 may include at least one plus anti-asphyxia valve (AAV) 674 in proximity to the cushion 630, which allows CO₂ and other gases exhaled by the user to escape in the event that the vents (e.g., the vents 672 or 676) fail when the respiratory therapy device is active.

Referring back to FIG. 1, the conduit 140 (also referred to as an air circuit or tube) allows the flow of air between components of the respiratory therapy system 100, such as between the respiratory therapy device 110 and the user interface 120. In some implementations, there can be separate limbs of the conduit for inhalation and exhalation. In other implementations, a single limb conduit is used for both inhalation and exhalation.

Referring to FIG. 3A, the conduit 140 includes a first end 142 that is coupled to the air outlet 118 of the respiratory therapy device 110. The first end 142 can be coupled to the air outlet 118 of the respiratory therapy device 110 using a variety of techniques (e.g., a press fit connection, a snap fit connection, a threaded connection, etc.). In some implementations, the conduit 140 includes one or more heating elements that heat the pressurized air flowing through the conduit 140 (e.g., heat the air to a predetermined temperature or within a range of predetermined temperatures). Such heating elements can be coupled to and/or imbedded in the conduit 140. In such implementations, the first end 142 can include an electrical contact that is electrically coupled to the respiratory therapy device 110 to power the one or more heating elements of the conduit 140. For example, the electrical contact can be electrically coupled to an electrical contact of the air outlet 118 of the respiratory therapy device 110. In this example, electrical contact of the conduit 140 can be a male connector and the electrical contact of the air outlet 118 can be female connector, or, alternatively, the opposite configuration can be used.

The display device 150 is generally used to display image(s) including still images, video images, or both and/or information regarding the respiratory therapy device 110. For example, the display device 150 can provide information regarding the status of the respiratory therapy device 110 (e.g., whether the respiratory therapy device 110 is on/off, the pressure of the air being delivered by the respiratory therapy device 110, the temperature of the air being delivered by the respiratory therapy device 110, etc.) and/or other information (e.g., a sleep score and/or a therapy score, also referred to as a myAir™ score, such as described in WO 2016/061629 and U.S. Patent Pub. No. 2017/0311879, which are hereby incorporated by reference herein in their entireties, the current date/time, personal information for the user 20, etc.). In some implementations, the display device 150 acts as a human-machine interface (HMI) that includes a graphic user interface (GUI) configured to display the image(s) as an input interface. The display device 150 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 110.

The humidifier 160 is coupled to or integrated in the respiratory therapy device 110 and includes a reservoir 162 for storing water that can be used to humidify the pressurized air delivered from the respiratory therapy device 110. The humidifier 160 includes a one or more heating elements 164 to heat the water in the reservoir to generate water vapor. The humidifier 160 can be fluidly coupled to a water vapor inlet of the air pathway between the blower motor 114 and the air outlet 118, or can be formed in-line with the air pathway between the blower motor 114 and the air outlet 118. For example, as shown in FIG. 3, air flow from the air inlet 116 through the blower motor 114, and then through the humidifier 160 before exiting the respiratory therapy device 110 via the air outlet 118.

While the respiratory therapy system 100 has been described herein as including each of the respiratory therapy device 110, the user interface 120, the conduit 140, the display device 150, and the humidifier 160, more or fewer components can be included in a respiratory therapy system according to implementations of the present disclosure. For example, a first alternative respiratory therapy system includes the respiratory therapy device 110, the user interface 120, and the conduit 140. As another example, a second alternative system includes the respiratory therapy device 110, the user interface 120, and the conduit 140, and the display device 150. Thus, various respiratory therapy systems can be formed using any portion or portions of the components shown and described herein and/or in combination with one or more other components.

The control system 200 includes one or more processors 202 (hereinafter, processor 202). The control system 200 is generally used to control (e.g., actuate) the various components of the system 10 and/or analyze data obtained and/or generated by the components of the system 10. The processor 202 can be a general or special purpose processor or microprocessor. While one processor 202 is illustrated in FIG. 1, the control system 200 can include any number of processors (e.g., one processor, two processors, five processors, ten processors, etc.) that can be in a single housing, or located remotely from each other. The control system 200 (or any other control system) or a portion of the control system 200 such as the processor 202 (or any other processor(s) or portion(s) of any other control system), can be used to carry out one or more steps of any of the methods described and/or claimed herein. The control system 200 can be coupled to and/or positioned within, for example, a housing of the user device 260, a portion (e.g., the respiratory therapy device 110) of the respiratory therapy system 100, and/or within a housing of one or more of the sensors 210. The control system 200 can be centralized (within one such housing) or decentralized (within two or more of such housings, which are physically distinct). In such implementations including two or more housings containing the control system 200, the housings can be located proximately and/or remotely from each other.

The memory device 204 stores machine-readable instructions that are executable by the processor 202 of the control system 200. The memory device 204 can be any suitable computer readable storage device or media, such as, for example, a random or serial access memory device, a hard drive, a solid state drive, a flash memory device, etc. While one memory device 204 is shown in FIG. 1, the system 10 can include any suitable number of memory devices 204 (e.g., one memory device, two memory devices, five memory devices, ten memory devices, etc.). The memory device 204 can be coupled to and/or positioned within a housing of a respiratory therapy device 110 of the respiratory therapy system 100, within a housing of the user device 260, within a housing of one or more of the sensors 210, or any combination thereof. Like the control system 200, the memory device 204 can be centralized (within one such housing) or decentralized (within two or more of such housings, which are physically distinct).

In some implementations, the memory device 204 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

As described herein, the processor 202 and/or memory device 204 can receive data (e.g., physiological data and/or audio data) from the one or more sensors 210 such that the data for storage in the memory device 204 and/or for analysis by the processor 202. The processor 202 and/or memory device 204 can communicate with the one or more sensors 210 using a wired connection or a wireless connection (e.g., using an RF communication protocol, a Wi-Fi communication protocol, a Bluetooth communication protocol, over a cellular network, etc.). In some implementations, the system 10 can include an antenna, a receiver (e.g., an RF receiver), a transmitter (e.g., an RF transmitter), a transceiver, or any combination thereof. Such components can be coupled to or integrated a housing of the control system 200 (e.g., in the same housing as the processor 202 and/or memory device 204), or the user device 260.

Referring to back to FIG. 1, the one or more sensors 210 include a pressure sensor 212, a flow rate sensor 214, temperature sensor 216, a motion sensor 218, a microphone 220, a speaker 222, a radio-frequency (RF) receiver 226, a RF transmitter 228, a camera 232, an infrared sensor 234, a photoplethysmogram (PPG) sensor 236, an electrocardiogram (ECG) sensor 238, an electroencephalography (EEG) sensor 240, a capacitive sensor 242, a force sensor 244, a strain gauge sensor 246, an electromyography (EMG) sensor 248, an oxygen sensor 250, an analyte sensor 252, a moisture sensor 254, a LiDAR sensor 256, or any combination thereof. Generally, each of the one or more sensors 210 are configured to output sensor data that is received and stored in the memory device 204 or one or more other memory devices.

While the one or more sensors 210 are shown and described as including each of the pressure sensor 212, the flow rate sensor 214, the temperature sensor 216, the motion sensor 218, the microphone 220, the speaker 222, the RF receiver 226, the RF transmitter 228, the camera 232, the infrared sensor 234, the photoplethysmogram (PPG) sensor 236, the electrocardiogram (ECG) sensor 238, the electroencephalography (EEG) sensor 240, the capacitive sensor 242, the force sensor 244, the strain gauge sensor 246, the electromyography (EMG) sensor 248, the oxygen sensor 250, the analyte sensor 252, the moisture sensor 254, and the LiDAR sensor 256, more generally, the one or more sensors 210 can include any combination and any number of each of the sensors described and/or shown herein.

As described herein, the system 10 generally can be used to generate physiological data associated with a user (e.g., a user of the respiratory therapy system 100) during a sleep session. The physiological data can be analyzed to generate one or more sleep-related parameters, which can include any parameter, measurement, etc. related to the user during the sleep session. The one or more sleep-related parameters that can be determined for the user 20 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 110, a heart rate, a heart rate variability, movement of the user 20, temperature, EEG activity, EMG activity, arousal, snoring, choking, coughing, whistling, wheezing, or any combination thereof.

The one or more sensors 210 can be used to generate, for example, physiological data, audio data, or both. Physiological data generated by one or more of the sensors 210 can be used by the control system 200 to determine a sleep-wake signal associated with the user 20 (FIG. 2) during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 210, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 210 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 110, or any combination thereof during the sleep session. The event(s) can include snoring, apneas, central apneas, obstructive apneas, mixed apneas, hypopneas, a mask leak (e.g., from the user interface 120), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 210 can also be used to determine a respiration signal associated with a user during a sleep session. The respiration signal is generally indicative of respiration or breathing of the user during the sleep session. The respiration signal can be indicative of and/or analyzed to determine (e.g., using the control system 200) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleep stage, an apnea-hypopnea index (AHI), pressure settings of the respiratory therapy device 110, or any combination thereof. The one or more events can include snoring, apneas, central apneas, obstructive apneas, mixed apneas, hypopneas, a mask leak (e.g., from the user interface 120), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 210, or from other types of data.

The pressure sensor 212 outputs pressure data that can be stored in the memory device 204 and/or analyzed by the processor 202 of the control system 200. In some implementations, the pressure sensor 212 is an air pressure sensor (e.g., barometric pressure sensor) that generates sensor data indicative of the respiration (e.g., inhaling and/or exhaling) of the user of the respiratory therapy system 100 and/or ambient pressure. In such implementations, the pressure sensor 212 can be coupled to or integrated in the respiratory therapy device 110. The pressure sensor 212 can be, for example, a capacitive sensor, an electromagnetic sensor, a piezoelectric sensor, a strain-gauge sensor, an optical sensor, a potentiometric sensor, or any combination thereof.

The flow rate sensor 214 outputs flow rate data that can be stored in the memory device 204 and/or analyzed by the processor 202 of the control system 200. Examples of flow rate sensors (such as, for example, the flow rate sensor 214) are described in International Publication No. WO 2012/012835 and U.S. Pat. No. 10,328,219, both of which are hereby incorporated by reference herein in their entireties. In some implementations, the flow rate sensor 214 is used to determine an air flow rate from the respiratory therapy device 110, an air flow rate through the conduit 140, an air flow rate through the user interface 120, or any combination thereof. In such implementations, the flow rate sensor 214 can be coupled to or integrated in the respiratory therapy device 110, the user interface 120, or the conduit 140. The flow rate sensor 214 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 214 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In some examples, the pressure sensor 212 can be used to determine a blood pressure of a user.

The temperature sensor 216 outputs temperature data that can be stored in the memory device 204 and/or analyzed by the processor 202 of the control system 200. In some implementations, the temperature sensor 216 generates temperatures data indicative of a core body temperature of the user 20 (FIG. 2), a skin temperature of the user 20, a temperature of the air flowing from the respiratory therapy device 110 and/or through the conduit 140, a temperature in the user interface 120, an ambient temperature, or any combination thereof. The temperature sensor 216 can be, for example, a thermocouple sensor, a thermistor sensor, a silicon band gap temperature sensor or semiconductor-based sensor, a resistance temperature detector, or any combination thereof.

The motion sensor 218 outputs motion data that can be stored in the memory device 204 and/or analyzed by the processor 202 of the control system 200. The motion sensor 218 can be used to detect movement of the user 20 during the sleep session, and/or detect movement of any of the components of the respiratory therapy system 100, such as the respiratory therapy device 110, the user interface 120, or the conduit 140. The motion sensor 218 can include one or more inertial sensors, such as accelerometers, gyroscopes, and magnetometers. In some implementations, the motion sensor 218 alternatively or additionally generates one or more signals representing bodily movement of the user, from which may be obtained a signal representing a sleep state of the user; for example, via a respiratory movement of the user. In some implementations, the motion data from the motion sensor 218 can be used in conjunction with additional data from another one of the sensors 210 to determine the sleep state of the user.

The microphone 220 outputs sound and/or audio data that can be stored in the memory device 204 and/or analyzed by the processor 202 of the control system 200. The audio data generated by the microphone 220 is reproducible as one or more sound(s) during a sleep session (e.g., sounds from the user 20). The audio data form the microphone 220 can also be used to identify (e.g., using the control system 200) an event experienced by the user during the sleep session, as described in further detail herein. The microphone 220 can be coupled to or integrated in the respiratory therapy device 110, the user interface 120, the conduit 140, or the user device 260. In some implementations, the system 10 includes a plurality of microphones (e.g., two or more microphones and/or an array of microphones with beamforming) such that sound data generated by each of the plurality of microphones can be used to discriminate the sound data generated by another of the plurality of microphones The speaker 222 outputs sound waves that are audible to a user of the system 10 (e.g., the user 20 of FIG. 2). The speaker 222 can be used, for example, as an alarm clock or to play an alert or message to the user 20 (e.g., in response to an event). In some implementations, the speaker 222 can be used to communicate the audio data generated by the microphone 220 to the user. The speaker 222 can be coupled to or integrated in the respiratory therapy device 110, the user interface 120, the conduit 140, or the user device 260.

The microphone 220 and the speaker 222 can be used as separate devices. In some implementations, the microphone 220 and the speaker 222 can be combined into an acoustic sensor 224 (e.g., a SONAR sensor), as described in, for example, WO 2018/050913, WO 2020/104465, U.S. Pat. App. Pub. No. 2022/0007965, each of which is hereby incorporated by reference herein in its entirety. In such implementations, the speaker 222 generates or emits sound waves at a predetermined interval and the microphone 220 detects the reflections of the emitted sound waves from the speaker 222. The sound waves generated or emitted by the speaker 222 have a frequency that is not audible to the human ear (e.g., below 20 Hz or above around 18 kHz) so as not to disturb the sleep of the user 20 or the bed partner 30 (FIG. 2). Based at least in part on the data from the microphone 220 and/or the speaker 222, the control system 200 can determine a location of the user 20 (FIG. 2) and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 110, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 210 include (i) a first microphone that is the same as, or similar to, the microphone 220, and is integrated in the acoustic sensor 224 and (ii) a second microphone that is the same as, or similar to, the microphone 220, but is separate and distinct from the first microphone that is integrated in the acoustic sensor 224.

The RF transmitter 228 generates and/or emits radio waves having a predetermined frequency and/or a predetermined amplitude (e.g., within a high frequency band, within a low frequency band, long wave signals, short wave signals, etc.). The RF receiver 226 detects the reflections of the radio waves emitted from the RF transmitter 228, and this data can be analyzed by the control system 200 to determine a location of the user and/or one or more of the sleep-related parameters described herein. An RF receiver (either the RF receiver 226 and the RF transmitter 228 or another RF pair) can also be used for wireless communication between the control system 200, the respiratory therapy device 110, the one or more sensors 210, the user device 260, or any combination thereof. While the RF receiver 226 and RF transmitter 228 are shown as being separate and distinct elements in FIG. 1, in some implementations, the RF receiver 226 and RF transmitter 228 are combined as a part of an RF sensor 230 (e.g. a RADAR sensor). In some such implementations, the RF sensor 230 includes a control circuit. The format of the RF communication can be Wi-Fi, Bluetooth, or the like.

In some implementations, the RF sensor 230 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 230. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 232 outputs image data reproducible as one or more images (e.g., still images, video images, thermal images, or any combination thereof) that can be stored in the memory device 204. The image data from the camera 232 can be used by the control system 200 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 232 can be used to, for example, identify a location of the user, to determine chest movement of the user (FIG. 2), to determine air flow of the mouth and/or nose of the user, to determine a time when the user enters the bed (FIG. 2), and to determine a time when the user exits the bed. In some implementations, the camera 232 includes a wide angle lens or a fish eye lens.

The infrared (IR) sensor 234 outputs infrared image data reproducible as one or more infrared images (e.g., still images, video images, or both) that can be stored in the memory device 204. The infrared data from the IR sensor 234 can be used to determine one or more sleep-related parameters during a sleep session, including a temperature of the user 20 and/or movement of the user 20. The IR sensor 234 can also be used in conjunction with the camera 232 when measuring the presence, location, and/or movement of the user 20. The IR sensor 234 can detect infrared light having a wavelength between about 700 nm and about 1 mm, for example, while the camera 232 can detect visible light having a wavelength between about 380 nm and about 740 nm.

The PPG sensor 236 outputs physiological data associated with the user 20 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 236 can be worn by the user 20, embedded in clothing and/or fabric that is worn by the user 20, embedded in and/or coupled to the user interface 120 and/or its associated headgear (e.g., straps, etc.), etc.

The ECG sensor 238 outputs physiological data associated with electrical activity of the heart of the user 20. In some implementations, the ECG sensor 238 includes one or more electrodes that are positioned on or around a portion of the user 20 during the sleep session. The physiological data from the ECG sensor 238 can be used, for example, to determine one or more of the sleep-related parameters described herein.

The EEG sensor 240 outputs physiological data associated with electrical activity of the brain of the user 20. In some implementations, the EEG sensor 240 includes one or more electrodes that are positioned on or around the scalp of the user 20 during the sleep session. The physiological data from the EEG sensor 240 can be used, for example, to determine a sleep state and/or a sleep stage of the user 20 at any given time during the sleep session. In some implementations, the EEG sensor 240 can be integrated in the user interface 120 and/or the associated headgear (e.g., straps, etc.).

The capacitive sensor 242, the force sensor 244, and the strain gauge sensor 246 output data that can be stored in the memory device 204 and used/analyzed by the control system 200 to determine, for example, one or more of the sleep-related parameters described herein. The EMG sensor 248 outputs physiological data associated with electrical activity produced by one or more muscles. The oxygen sensor 250 outputs oxygen data indicative of an oxygen concentration of gas (e.g., in the conduit 140 or at the user interface 120). The oxygen sensor 250 can be, for example, an ultrasonic oxygen sensor, an electrical oxygen sensor, a chemical oxygen sensor, an optical oxygen sensor, a pulse oximeter (e.g., SpO₂ sensor), or any combination thereof.

The analyte sensor 252 can be used to detect the presence of an analyte in the exhaled breath of the user 20. The data output by the analyte sensor 252 can be stored in the memory device 204 and used by the control system 200 to determine the identity and concentration of any analytes in the breath of the user. In some implementations, the analyte sensor 174 is positioned near a mouth of the user to detect analytes in breath exhaled from the user's mouth. For example, when the user interface 120 is a facial mask that covers the nose and mouth of the user, the analyte sensor 252 can be positioned within the facial mask to monitor the user's mouth breathing. In other implementations, such as when the user interface 120 is a nasal mask or a nasal pillow mask, the analyte sensor 252 can be positioned near the nose of the user to detect analytes in breath exhaled through the user's nose. In still other implementations, the analyte sensor 252 can be positioned near the user's mouth when the user interface 120 is a nasal mask or a nasal pillow mask. In this implementation, the analyte sensor 252 can be used to detect whether any air is inadvertently leaking from the user's mouth and/or the user interface 120. In some implementations, the analyte sensor 252 is a volatile organic compound (VOC) sensor that can be used to detect carbon-based chemicals or compounds. In some implementations, the analyte sensor 174 can also be used to detect whether the user is breathing through their nose or mouth. For example, if the data output by an analyte sensor 252 positioned near the mouth of the user or within the facial mask (e.g., in implementations where the user interface 120 is a facial mask) detects the presence of an analyte, the control system 200 can use this data as an indication that the user is breathing through their mouth.

The moisture sensor 254 outputs data that can be stored in the memory device 204 and used by the control system 200. The moisture sensor 254 can be used to detect moisture in various areas surrounding the user (e.g., inside the conduit 140 or the user interface 120, near the user's face, near the connection between the conduit 140 and the user interface 120, near the connection between the conduit 140 and the respiratory therapy device 110, etc.). Thus, in some implementations, the moisture sensor 254 can be coupled to or integrated in the user interface 120 or in the conduit 140 to monitor the humidity of the pressurized air from the respiratory therapy device 110. In other implementations, the moisture sensor 254 is placed near any area where moisture levels need to be monitored. The moisture sensor 254 can also be used to monitor the humidity of the ambient environment surrounding the user, for example, the air inside the bedroom.

The Light Detection and Ranging (LiDAR) sensor 256 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 256 can measure and map an area extending 5 meters or more away from the sensor. The LiDAR data can be fused with point cloud data estimated by an electromagnetic RADAR sensor, for example. The LiDAR sensor(s) 256 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 210 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 210 can be integrated in and/or coupled to any one or more of the components of the system 100, including the respiratory therapy device 110, the user interface 120, the conduit 140, the humidifier 160, the control system 200, the user device 260, the activity tracker 270, or any combination thereof. For example, the microphone 220 and the speaker 222 can be integrated in and/or coupled to the user device 260 and the pressure sensor 212 and/or flow rate sensor 132 are integrated in and/or coupled to the respiratory therapy device 110. In some implementations, at least one of the one or more sensors 210 is not coupled to the respiratory therapy device 110, the control system 200, or the user device 260, and is positioned generally adjacent to the user 20 during the sleep session (e.g., positioned on or in contact with a portion of the user 20, worn by the user 20, coupled to or positioned on the nightstand, coupled to the mattress, coupled to the ceiling, etc.).

One or more of the respiratory therapy device 110, the user interface 120, the conduit 140, the display device 150, and the humidifier 160 can contain one or more sensors (e.g., a pressure sensor, a flow rate sensor, or more generally any of the other sensors 210 described herein). These one or more sensors can be used, for example, to measure the air pressure and/or flow rate of pressurized air supplied by the respiratory therapy device 110.

The data from the one or more sensors 210 can be analyzed (e.g., by the control system 200) to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 210, or from other types of data.

The user device 260 (FIG. 1) includes a display device 262. The user device 260 can be, for example, a mobile device such as a smart phone, a tablet, a gaming console, a smart watch, a laptop, or the like. Alternatively, the user device 260 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 262 is generally used to display image(s) including still images, video images, or both. In some implementations, the display device 262 acts as a human-machine interface (HMI) that includes a graphic user interface (GUI) configured to display the image(s) and an input interface. The display device 262 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 260. In some implementations, one or more user devices can be used by and/or included in the system 10.

In some implementations, the system 100 also includes an activity tracker 270. The activity tracker 270 is generally used to aid in generating physiological data associated with the user. The activity tracker 270 can include one or more of the sensors 210 described herein, such as, for example, the motion sensor 138 (e.g., one or more accelerometers and/or gyroscopes), the PPG sensor 154, and/or the ECG sensor 156. The physiological data from the activity tracker 270 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 270 is coupled (e.g., electronically or physically) to the user device 260.

In some implementations, the activity tracker 270 is a wearable device that can be worn by the user, such as a smartwatch, a wristband, a ring, or a patch. For example, referring to FIG. 2, the activity tracker 270 is worn on a wrist of the user 20. The activity tracker 270 can also be coupled to or integrated a garment or clothing that is worn by the user. Alternatively still, the activity tracker 270 can also be coupled to or integrated in (e.g., within the same housing) the user device 260. More generally, the activity tracker 270 can be communicatively coupled with, or physically integrated in (e.g., within a housing), the control system 200, the memory device 204, the respiratory therapy system 100, and/or the user device 260.

In some implementations, the system 100 also includes a blood pressure device 280. The blood pressure device 280 is generally used to aid in generating cardiovascular data for determining one or more blood pressure measurements associated with the user 20. The blood pressure device 280 can include at least one of the one or more sensors 210 to measure, for example, a systolic blood pressure component and/or a diastolic blood pressure component.

In some implementations, the blood pressure device 280 is a sphygmomanometer including an inflatable cuff that can be worn by the user 20 and a pressure sensor (e.g., the pressure sensor 212 described herein). For example, in the example of FIG. 2, the blood pressure device 280 can be worn on an upper arm of the user 20. In such implementations where the blood pressure device 280 is a sphygmomanometer, the blood pressure device 280 also includes a pump (e.g., a manually operated bulb) for inflating the cuff. In some implementations, the blood pressure device 280 is coupled to the respiratory therapy device 110 of the respiratory therapy system 100, which in turn delivers pressurized air to inflate the cuff. More generally, the blood pressure device 280 can be communicatively coupled with, and/or physically integrated in (e.g., within a housing), the control system 200, the memory device 204, the respiratory therapy system 100, the user device 260, and/or the activity tracker 270.

In other implementations, the blood pressure device 280 is an ambulatory blood pressure monitor communicatively coupled to the respiratory therapy system 100. An ambulatory blood pressure monitor includes a portable recording device attached to a belt or strap worn by the user 20 and an inflatable cuff attached to the portable recording device and worn around an arm of the user 20. The ambulatory blood pressure monitor is configured to measure blood pressure between about every fifteen minutes to about thirty minutes over a 24-hour or a 48-hour period. The ambulatory blood pressure monitor may measure heart rate of the user 20 at the same time. These multiple readings are averaged over the 24-hour period. The ambulatory blood pressure monitor determines any changes in the measured blood pressure and heart rate of the user 20, as well as any distribution and/or trending patterns of the blood pressure and heart rate data during a sleeping period and an awakened period of the user 20. The measured data and statistics may then be communicated to the respiratory therapy system 100.

The blood pressure device 280 maybe positioned external to the respiratory therapy system 100, coupled directly or indirectly to the user interface 120, coupled directly or indirectly to a headgear associated with the user interface 120, or inflatably coupled to or about a portion of the user 20. The blood pressure device 280 is generally used to aid in generating physiological data for determining one or more blood pressure measurements associated with a user, for example, a systolic blood pressure component and/or a diastolic blood pressure component. In some implementations, the blood pressure device 280 is a sphygmomanometer including an inflatable cuff that can be worn by a user and a pressure sensor (e.g., the pressure sensor 212 described herein).

In some implementations, the blood pressure device 280 is an invasive device which can continuously monitor arterial blood pressure of the user 20 and take an arterial blood sample on demand for analyzing gas of the arterial blood. In some other implementations, the blood pressure device 280 is a continuous blood pressure monitor, using a radio frequency sensor and capable of measuring blood pressure of the user 20 once very few seconds (e.g., every 3 seconds, every 5 seconds, every 7 seconds, etc.) The radio frequency sensor may use continuous wave, frequency-modulated continuous wave (FMCW with ramp chirp, triangle, sinewave), other schemes such as PSK, FSK etc., pulsed continuous wave, and/or spread in ultra wideband ranges (which may include spreading, PRN codes or impulse systems).

While the control system 200 and the memory device 204 are described and shown in FIG. 1 as being a separate and distinct component of the system 100, in some implementations, the control system 200 and/or the memory device 204 are integrated in the user device 260 and/or the respiratory therapy device 110. Alternatively, in some implementations, the control system 200 or a portion thereof (e.g., the processor 202) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system according to implementations of the present disclosure. For example, a first alternative system includes the control system 200, the memory device 204, and at least one of the one or more sensors 210 and does not include the respiratory therapy system 100. As another example, a second alternative system includes the control system 200, the memory device 204, at least one of the one or more sensors 210, and the user device 260. As yet another example, a third alternative system includes the control system 200, the memory device 204, the respiratory therapy system 100, at least one of the one or more sensors 210, and the user device 260. Thus, various systems can be formed using any portion or portions of the components shown and described herein and/or in combination with one or more other components.

As used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 262 of the user device 260 (FIG. 1) to manually initiate or terminate the sleep session.

Generally, the sleep session includes any point in time after the user 20 has laid or sat down in the bed 40 (or another area or object on which they intend to sleep), and has turned on the respiratory therapy device 110 and donned the user interface 120. The sleep session can thus include time periods (i) when the user 20 is using the respiratory therapy system 100, but before the user 20 attempts to fall asleep (for example when the user 20 lays in the bed 40 reading a book); (ii) when the user 20 begins trying to fall asleep but is still awake; (iii) when the user 20 is in a light sleep (also referred to as stage 1 and stage 2 of non-rapid eye movement (NREM) sleep); (iv) when the user 20 is in a deep sleep (also referred to as slow-wave sleep, SWS, or stage 3 of NREM sleep); (v) when the user 20 is in rapid eye movement (REM) sleep; (vi) when the user 20 is periodically awake between light sleep, deep sleep, or REM sleep; or (vii) when the user 20 wakes up and does not fall back asleep.

The sleep session is generally defined as ending once the user 20 removes the user interface 120, turns off the respiratory therapy device 110, and gets out of bed 40. In some implementations, the sleep session can include additional periods of time, or can be limited to only some of the above-disclosed time periods. For example, the sleep session can be defined to encompass a period of time beginning when the respiratory therapy device 110 begins supplying the pressurized air to the airway or the user 20, ending when the respiratory therapy device 110 stops supplying the pressurized air to the airway of the user 20, and including some or all of the time points in between, when the user 20 is asleep or awake.

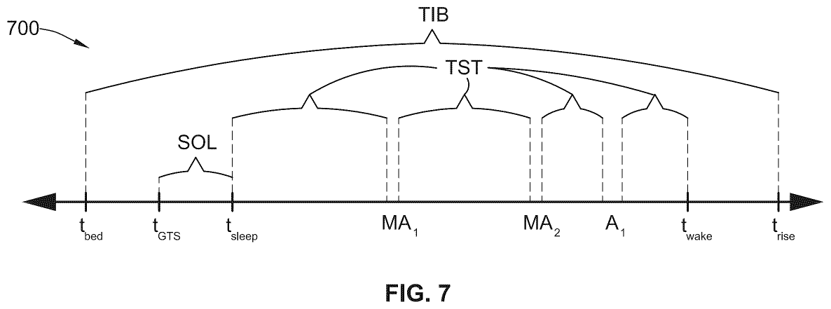

Referring to the timeline 700 in FIG. 7 the enter bed time t_{bed} is associated with the time that the user initially enters the bed (e.g., bed 40 in FIG. 2) prior to falling asleep (e.g., when the user lies down or sits in the bed). The enter bed time t_{bed} can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 260, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 700 of FIG. 7, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

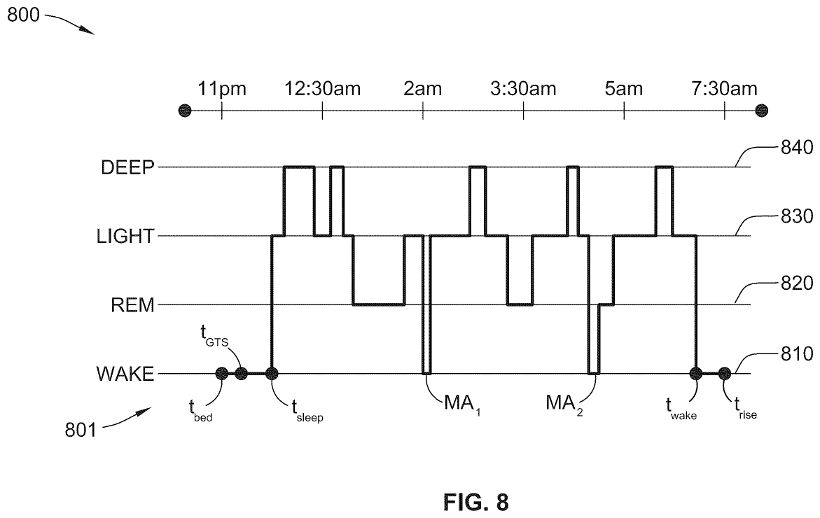

Referring to FIG. 8, an exemplary hypnogram 800 corresponding to the timeline 700 (FIG. 7), according to some implementations, is illustrated. As shown, the hypnogram 800 includes a sleep-wake signal 801, a wakefulness stage axis 810, a REM stage axis 820, a light sleep stage axis 830, and a deep sleep stage axis 840. The intersection between the sleep-wake signal 801 and one of the axes 810-840 is indicative of the sleep stage at any given time during the sleep session.

The sleep-wake signal 801 can be generated based on physiological data associated with the user (e.g., generated by one or more of the sensors 210 described herein). The sleep-wake signal can be indicative of one or more sleep states, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 800 is shown in FIG. 8 as including the light sleep stage axis 830 and the deep sleep stage axis 840, in some implementations, the hypnogram 800 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration ratio, a number of events per hour, a pattern of events, or any combination thereof. Information describing the sleep-wake signal can be stored in the memory device 204.

The hypnogram 800 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 7), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.) The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 7), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 210 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based on, for example, data generated by the motion sensor 218, the microphone 220, the camera 232, or any combination thereof. The go-to-sleep time can be determined based on, for example, data from the motion sensor 218 (e.g., data indicative of no movement by the user), data from the camera 232 (e.g., data indicative of no movement by the user and/or that the user has turned off the lights) data from the microphone 220 (e.g., data indicative of the using turning off a TV), data from the user device 260 (e.g., data indicative of the user no longer using the user device 260), data from the pressure sensor 212 and/or the flow rate sensor 214 (e.g., data indicative of the user turning on the respiratory therapy device 110, data indicative of the user donning the user interface 120, etc.), or any combination thereof.

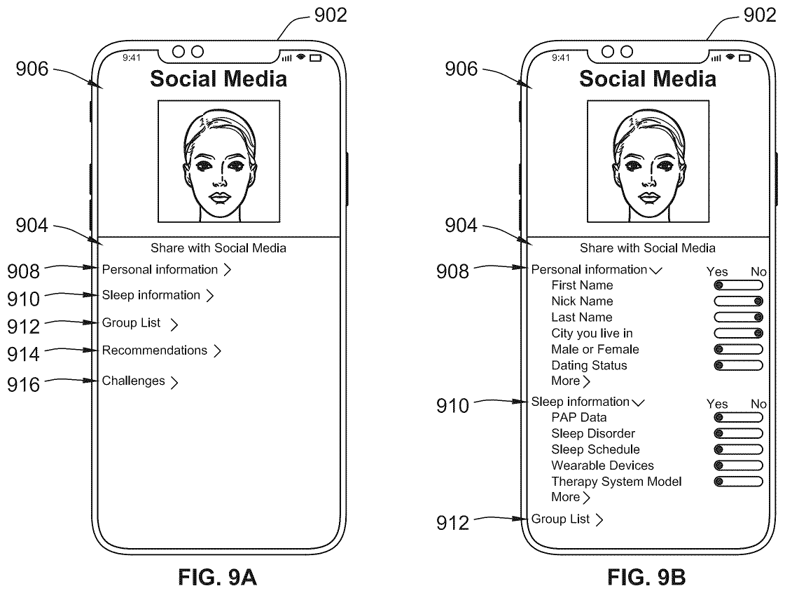

While the discussion of FIGS. 1 to 8 provide detail regarding sleep scores and respiratory therapy devices, the discussion of FIGS. 9 to 14 provide additional detail regarding systems and methods for matching a user to a user group based on sleep information and/or personal information of the user.

FIGS. 9A and 9B depict a user device 902 presenting a personal homepage of a user 906 of a sleep community application, according to some implementations of the present disclosure. The user device 902 presents the personal homepage of the user 906 via a graphical user interface 904 of the user device 902. The user device 902 can be of any suitable type, such as a smartphone, a tablet computer, a desktop computer, a laptop computer, a smartwatch, a personal digital assistant (PDA), a respiratory therapy device, a sleep enhancement or therapy device, etc.

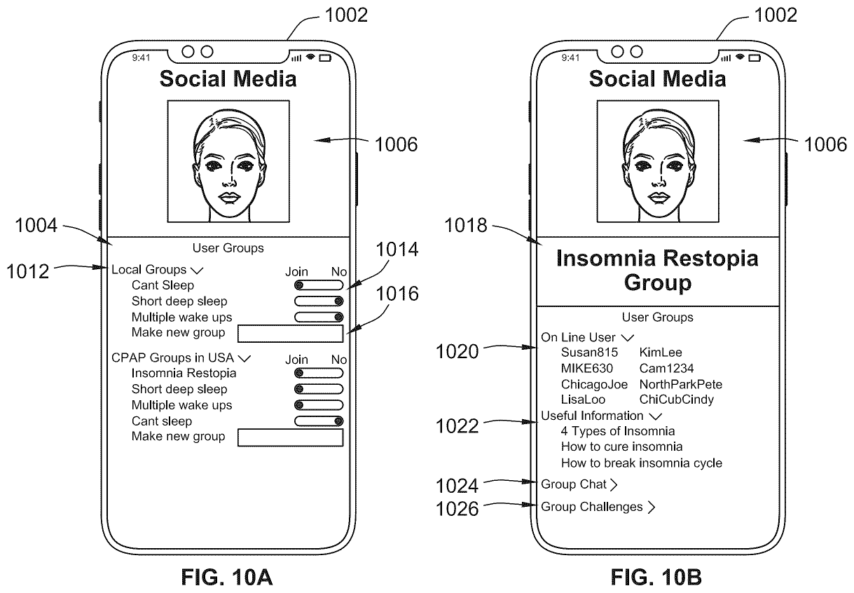

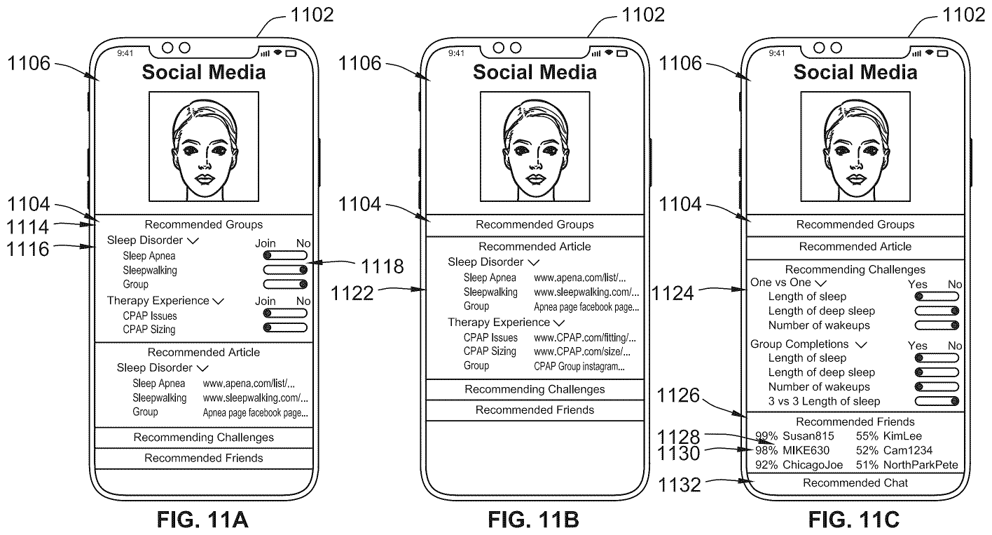

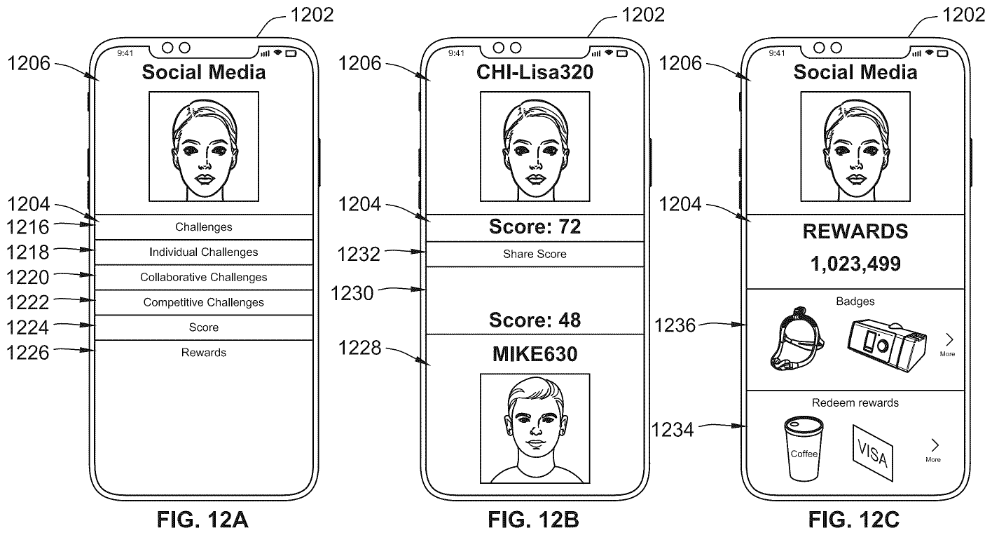

In FIG. 9A, the user device 902 is presenting a personal home page of the sleep community application. On the homepage, the first user 906 is able to view, for example, the first user's 906 own personal information 908 and sleep information 910. According to some embodiments, the homepage may also include access to about a list of user groups 912 that the first user 906 is a member of, customized recommendations 914 based on the sleep/personal information of the first user 906, and challenges 916 that could help improve the first user's sleep habits and sleep health. As described in more detail with respect to FIGS. 10A and 10B, expansion of the group list tab 912 allows the user to view groups of which the user is a member. As described in more detail with respect to FIGS. 11A through 11C, expansion of the recommendations tab 914 allows the user to view a list of recommended groups, challenges, other users, and suitable media contents (e.g., videos, podcasts, articles, etc.) based on the sleep information and/or personal information of the first user. As described in more detail with respect to FIGS. 12A and 12C, expansion of the challenges tab 916 allows the user to select a variety of participatory challenges that involve sleep therapy or respiratory therapy.

In FIG. 9B, the tabs containing the first user's personal information 908 and sleep information 910 are expanded to show an example of some personal information and sleep information that can be collected. The sleep community application generally matches the first user 906 to a user group based on sleep information 910. For example, the sleep information can include a sleep diagnosis (e.g., of insomnia, restless leg syndrome, parasomnia, narcolepsy, circadian rhythm sleep-wake disorders, sleep apnea, etc.), a sleep schedule, a duration of sleep, restlessness, severity of sleep disorder, user device usage, use of a type of sleep enhancement device (e.g., sleep enhancement headband), etc. In some embodiment, the sleep information can also include respiratory information. The respiratory information can include any suitable categories. For example, the respiratory information can include use of a respiratory therapy system, a type of respiratory therapy device, a duration of respiratory therapy usage (e.g., how many hours per night a user uses their respiratory therapy system), a length of respiratory therapy usage (e.g., for what period of time, such as days, months, and/or years, a user has been using their respiratory therapy system), a sleep schedule, a duration of sleep, medical conditions, an apnea hypopnea index, a sleep score, etc. The respiratory therapy device can be of any suitable type, such as positive airway pressure (PAP) device or non-PAP alternative treatment device (e.g., mandibular advancement appliance, positional therapy device, oral muscle training tool, etc.). According to some embodiments, the application is able to match the first user 906 to a user group based on personal information 908 of the first user 906. According to some other embodiments, a user group or user group search based on the sleep information 910 of the first user 906 can be further refined and/or customized based on one or more features from the personal information 908 of the first user 906. The personal information 908 can include any suitable categories. For example, the personal information can include age, gender identity, sex, sexual orientation, location, interests, hobbies, likes, dislikes, preferences, lifestyle choices (e.g., smoker, alcohol use, dietary), etc.

In some embodiments, the first user 906 is capable of entering, removing, and/or modifying the personal information 906 and the sleep information 908 associated with the first user 906 via the application (e.g., via one or more views of the graphical user interface 904). In addition to the first user 906 providing information (e.g., sleep information, respiratory information, and personal information), in some embodiments, some, or all, of this information can be provided automatically for, and possibly after approval by, the first user 906. For example, an external device can provide this information to the application. The external device can be any device that includes (whether by user input or inference) information about the user. For example, the external device can include the user device 902, a sleep enhancement device, a respiratory therapy device (e.g., a positive air-pressure machine), a nearable device, a wearable monitoring device, an airable device, calendars, etc.

FIGS. 10A and 10B depict a user device 1002 presenting a list of user groups that the first user 1006 is a member of and a landing page for one of the user groups, according to some implementations of the present disclosure. FIG. 10A shows the homepage of the first user 1006 being centered on the list of user groups 1012 of which the first user 1006 is a member. According to some embodiments, the first user 1006 may be automatically assigned to some or all the user groups 1012 based on the sleep information and/or personal information entered by the first user 1006. Additionally, or alternatively, the application may recommend a user group and present the first user 1006 with a choice on whether to join the recommended user group. For example, the application may display a toggle button 1014 next to the user group 1012. The first user 1006 may select the toggle button 1014 to join the user group 1012 or deselect the toggle button 1014 to disassociate from the user group. According to some embodiments, the application may also have an option to create a new user group based on a feature present in the sleep information or personal information of the first user 1006. For example, responsive to the application determining that the sleep information or personal information of the first user 1006 does not match any preexisting user groups, the application may automatically define and generate a new user group based on a feature found in the sleep information or personal information of the first user 1006. Alternatively, the application may simultaneously identify one or more features from the sleep information or personal information of the first user 1006 and automatically generate one or more new user groups based on the one or more features. Additionally, or alternatively, the application may display an option button 1016 in the graphical user interface 1004 for the first user 1006 to freely create a new group. Upon selecting the option button 1016, the application may prompt the first user 1006 to identify one or more features from his/her sleep information or personal information to base the new group on.

Next, responsive to identifying one or more features found in the sleep information or personal information of the first user 1006, the application may automatically induct the first user 1006 to the one or more newly created user groups or prompt the first user 1006 to join any of the new user groups. The application may also conduct a database search for other users who have the same feature in their personal information or sleep information. According to some embodiments, the application may automatically induct other users with the same feature into one or more of the new user groups. Alternatively, the application may cause notifications to be sent to the other users to prompting them to join one or more of the new user groups.

FIG. 10B depicts a user interface presenting a landing page of a user group of which the first user 1006 is a member. For example, the user group may be dedicated to users who are insomniacs based on their sleep information (e.g., inferred based on their sleep information, self-identified by the users, etc.). The sleep information may be obtained through the users'voluntary identification or through automatic recognition via an external device or a monitoring device, such as a sleep enhancement system or a respiratory therapy system that detects the user as someone who chronically sleeps less than a certain amount of time each night. A key function of the group landing page is to provide a venue for users who cope with or have interest in a particular sleep-related issue to find support and social network among other similarly-positioned users. Through group discussion and/or other participatory activities (e.g., group challenges), users are motivated to take up or continue with sleep therapy that could improve their sleep health over time. According to some embodiments, the landing page can include a user group name 1018. The landing page can also include a list 1020 of users who are members of the user group based on their having a feature associated with the user group (e.g., having insomnia). The landing page can further include a compilation of useful information 1022 curated for the user group based on the group's common feature (e.g., insomnia). For example, the compilation of useful information 1022 with regard to a user group related to insomnia could include a list of suitable media content (e.g., a list of articles, podcasts, or videos, etc.) on medical knowledge about insomnia, website for insomnia support groups, sleep therapy products for treating insomnia, etc. Further, the landing page can include a group chat room 1024. For example, the group chat 1024 feature can include options that allow the first user 1006 to enter into a one-on-one chatroom with another user or a one-to-many discussion board with multiple other users of the user group. Further still, the landing page can include a list of group challenges 1026 that the users may participate in with each other. For example, the group challenges 1026 feature can include a list of recommended participatory games (i.e., challenges) that are relevant to the common feature of the user group. With regard to a user group related to insomnia, the list of recommended challenges may include challenges for the first user 1006 to follow a set sleep schedule, maintain a minimum daily exercise regimen, limit intake of caffeinated beverages for a certain number of days, etc. The challenge feature of the present disclosure is described in further detail with respect to FIG. 12A-12C.

FIG. 11A-11C depict a user device presenting recommendations to the first user 1106 based on the sleep information and/or personal information of the first user 1106, according to some implementations of the present disclosure. According to some examples, the list of recommendations 1114 may include sub-lists of recommendations for groups, suitable media content (e.g., articles, podcasts, videos, etc.), challenges, and connection with other users (e.g., recommended friends). FIG. 11A shows the homepage of the first user 1106. More specifically, FIG. 11A emphasizes on a sub-list of recommended user groups 1116 based on the sleep information and/or personal information of the first user 1106. According to some embodiments, the recommendations can also be based on other sources such as the performance of the first user 1106 in a challenge or the recommendation from a second user (e.g., a member of the same user group, a friended user, etc.). According to some embodiments, the list of recommendations 1114 also includes one or more buttons 1118 that enable the first user 1106 to join the one or more recommended user groups.

FIG. 11B depicts a continuation of the list of recommendations 1114. According to some embodiments, the list of recommendations 1114 may also include a sub-list of one or more recommended media content 1122 curated based on the sleep information and/or personal information of the first user 1106. For example, the recommended media content may include links to in-application or external articles, podcasts, or videos on medical treatment and therapy related to a sleeping condition that the first user 1106 has based on his/her sleeping information. Referring to the example in FIG. 11B, if the first user 1106 inputted sleep apnea into his/her sleep information, the list of recommended media content may show one or more articles, podcasts, or videos related to sleep apnea. According to some embodiments, a piece of media content could be identified as being related to one or more sleep-issues based on a tag, which could be curated by a person (e.g., a user or a system administrator) or by an algorithm (e.g., a keyword search algorithm).

FIG. 11C depicts a further continuation of the list of recommendations 1114. According to some embodiments, the list of recommendations 1114 may further include a sub-list of one or more recommended connections with other users based on a commonality between the first user 1106 and the other users. For example, the application may determine based on the sleep information and/or personal information of the first user 1106 and a second user 1128 that the users 1106 and 1128 have one or more commonalities with each other. According to some embodiments, the application can recommend the first user 1106 to make connection with the second user 1128 (e.g., adding the second user 1128 as a friend). According to some embodiments, the application may include a commonality indicator next to each recommended user. For example, a commonality indicator 1130 can be expressed as a percentage of information (e.g., sleep information, search criteria, respiratory information, personal information, etc.) that is common between the first user 1106 and the second user 1128.

According to some embodiments, the list of recommendations 1114 may further include a sub-list with one or more recommended participatory challenges 1124 that are recommended to the first user 1106 based on his/her sleep information and/or personal information. For example, the participatory challenges 1124 could include gamification of activities that encourage the first user 1106 to participate in sleep therapy that help with sleep issues experienced by the first user 1106. The participatory challenges 1124, for example, could include activities that assist the first user 1106 to adhere to a respiratory therapy. The challenge feature of the present disclosure is described in further detail with respect to FIG. 12A-12C.

According to some embodiments, the list of recommendations 1114 may further include a sub-list that recommends one or more forums 1132 (e.g., chatrooms or links to message posting websites) based on the sleep information and/or personal information of the first user 1106. The recommended forums 1132 provide the first user 1106 with venues to discuss his/her sleep therapy and sleep-related issues with other users who experience similar issues. According to some embodiments, the recommended forums include in-application chatrooms for the application's user community (as previously described with respect to FIG. 10B). Additionally, or alternatively, the recommended forums 1132 may also include links to third-party discussion boards that have been tagged (either by a user, a system administrator, or a keyword search algorithm) as being related to an issue that the first user 1106 has identified in his/her sleep information or respiratory information.

FIG. 12A-12C depict a user device 1202 presenting participatory challenges in which the first user 1206 can participate, according to some implementations of the present disclosure. Generally, the challenges are participatory activities related to sleep therapy (e.g., sleeping for a certain length each day, go to bed by certain time each day, number of consecutive days that a user has used the respiratory therapy system, etc.). By converting sleep therapy into fun games and involving participation by other similarly-positioned peers, users on sleep therapy are motivated to continue with their sleep therapy sessions.

FIG. 12A shows a challenges page 1216 where the first user 1206 has the option to participate in one or more challenges. According to some embodiments, the challenge page 1216 may include a selection of different types of challenges. According to some embodiments, the application may include one or more individual challenges 1218 undertaken by the first user 1206 alone. For example, the goal of a first challenge can be that the first user 1206 must increase usage of a respiratory therapy system (e.g., a positive airway pressure system) by a threshold number of hours. As another example, the goal of a second challenge can be that the first user must adopt a certain habit or behavioral change that improves sleep health (e.g., go to bed by a certain time, maintain a daily exercise regimen, limit the number of caffeinated drinks per day, or avoid food intake after a certain time). According to some embodiments, a change in the behavior of the first user 1206 could be measured by comparing an initial user score calculated at the start of the challenge to an end user score calculated at the end of the challenge. The performance of the first user 1206 could be compared to a static threshold (e.g., a fixed score or achievement, a duration of respiratory therapy usage over a period, etc.) or a dynamic threshold (e.g., the first user's 1206 past performance, an average performance of the user community, etc.).

The application can also include group challenges 1220 undertaken by the first user 1206 with at least one other user (e.g., a friended user or a second user from a user group of which the first user 1206 is a member). When the challenge is of a collaborative type, a score of the first user 1206 could be aggregated with the score of other participating users to determine whether the aggregate score exceed a certain threshold. For example, the goal of a collaborative group challenge involving the first user 1206 and a second user can be that both users must wear a respiratory therapy system for an average of at least seven hours per night for fourteen consecutive days.

According to some embodiments, the application may further include competitive challenges 1222. A competitive challenge 1222 may involve the first user 1206 competing against one or more other individual users. For example, a competitive challenge could be between the first user 1206 and a second user, wherein the users compete against each other for the highest sleep score over the course of a week. Alternatively, a competitive challenge may also be a group-against-group type, where the first user 1206 collaborates with one or more users of a same user group to compete against the users of another user group. For example, a user group of which the first user 1206 is a member may compete against another user group for the highest aggregate or average number of hours of sleep per night. According to some embodiments, the challenge page 1216 may further include one or more buttons for accessing the first user's score 1224 and the rewards 1226 received by the first user 1206. The score feature 1224 and the rewards feature 1226 are described in additional detail with respect to FIG. 12B.

FIG. 12B depicts a user device 1202 presenting a score/ranking page where the score 1230 of the first user 1206 is compared with that of one or more other users (e.g., a second user, a second group of users, etc.). According to some embodiments, the score 1230 could be an index calculated from the sleep information associated with the first user 1206 according to a metric. For example, a user score can be based on one or more measured or tracked parameters derived from sleep information or respiratory information collected by one or more of an external device, a sleep enhancement system, and a respiratory therapy system, such as abstract indexes calculated based on a metric (e.g., a sleep score), counters (e.g., a number of challenges completed, a number of times the first user 1206 performed tasks recommended by the application, a frequency that the first user 1206 uses the respiratory therapy system), or a timer/time stamp (e.g., a duration of respiratory therapy usage on a CPAP system or a time when the first performs a physical activity), etc. A respiratory therapy system can include one or more of a positive air-pressure machine, a wearable monitoring device, a nearable device, an airable device, etc. According to some embodiments, the score of users could be sorted and displayed in a ranking format. According to some embodiments, the score/ranking page may further include a button 1232 allowing the score 1230 and/or ranking of the first user 1206 to be shared to one or more user devices of one or more second users 1228 (e.g., a friended user, a user belonging to the same user group, etc.).

FIG. 12C depicts a graphical user interface 1204 presenting a rewards page where rewards received by, and accomplishments of, the first user 1206 are presented. According to some embodiments, the application presents a reward to the first user 1206 via the user device 1202 when the first user 1206 accomplishes a challenge or wins a challenge against one or more other users. For example, the rewards could include one or more digital icons (e.g., digital badges 1236) indicating the first user's accomplishments (e.g., consecutive use of a respiratory therapy system for two consecutive months, etc.). Additionally, or alternatively, if the first user 1206 achieved the goal, the first user can receive a certain number of points that can be used for redeeming a gift from a list of redeemable rewards 1234. For example, the redeemable rewards may include pecuniary reward, consumer items, respiratory therapy supplies, a membership, a service, or a charitable donation by a third party to a designated account. According to some embodiments, if the first user 1206 does not succeed in accomplishing a challenge (e.g., a score of the first user 1206 is lower than a threshold score for accomplishing the challenge), the application can present recommendations to the user via the user device 1202 to help the first user 1206 improve on future performance. For example, the application may recommend one or more pieces of sleep health media content (e.g., articles, podcasts, videos, etc.) related to the type of sleep therapy challenge that the first user 1206 failed to accomplish.

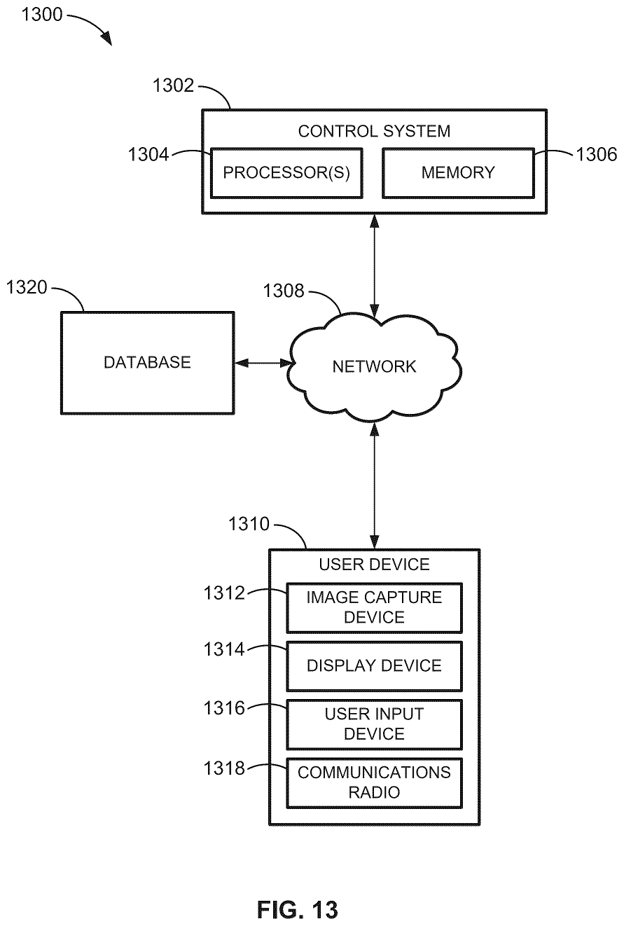

FIG. 13 is a block diagram of a system 1300 for assigning users to groups based on sleep information and/or personal information of the user, according to some implementations of the present disclosure. The system 1300 includes a control system 1302, a network 1308, and a user device 1310. In one embodiment, the user device 1310 is communicatively coupled to the control system 1302 via the network 1308. The network 1308 can be of any suitable type (e.g., a local area network and/or wide area network, such as the Internet). Accordingly, the network 1308 can include wired and/or wireless links. Though depicted as separate devices, in some embodiments, the control system 1302 can be resident on the user device 1310. In such embodiments, the user device 1310 may not need to be connected to, or transmit data via, the network 1308.

The control system 1302 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. The control system 1302 is configured (for example, by using corresponding programming as will be well understood by those skilled in the art) to carry out one or more of the steps, actions, and/or functions described herein.

By one optional approach the control system 1302 operably couples to a memory. The memory may be integral to the control system 1302 or can be physically discrete (in whole or in part) from the control system 1302 as desired. This memory can also be local with respect to the control system 1302 (where, for example, both share a common circuit board, chassis, power supply, and/or housing) or can be partially or wholly remote with respect to the control system 1302 (where, for example, the memory is physically located in another facility, metropolitan area, or even country as compared to the control system 1302).

This memory can serve, for example, to non-transitorily store the computer instructions that, when executed by the control system 1302, cause the control system 1302 to behave as described herein. As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as an erasable programmable read-only memory (EPROM).

The control system 1302 generally determines matches for users based on sleep information. For example, the control system 1302 can receive sleep information for users and determine matches based on the sleep information. The control system 1302 determines matches between users, based on the sleep information, and causes transmission of notifications based on the matches.

The user device 1310 generally provides sleep information and presents user interfaces of an application to a user. Accordingly, the user device 1310 can be of any suitable type. For example, the user device can be a smartphone, a smartwatch, a laptop or desktop computer, a personal digital assistant (PDA), a tablet computer, an automotive infotainment system, a smart mirror, a television, a respiratory therapy device, a sleep enhancement or therapy device, etc. In some embodiments, the user device 1310 includes an image capture device 1312, a display device 1314, a user input device 1316, and a communications radio 1318. The image capture device 1312 can be of any suitable type (e.g., a digital camera) and is configured to capture still and/or video images. The display device 1314 can be of any suitable type (e.g., LED display, LCD, etc.) and is configured to present information (e.g., instructions, a graphical user interface (GUI), images, etc.) to the user. The user input device 1316 can be of any suitable type (e.g., a keyboard, mouse, touchscreen, joystick, trackpad, microphone, etc.) and is configured to receive user input from the user. It should be noted that, in some embodiments, the display device 1314 and the user input device 1316 can be combined into a single device, such as a touchscreen. The communications radio 1318 can be of any suitable type (e.g., a near field communication (NFC) radio, a wireless wide area network (WWAN) radio, a Wi-Fi radio, etc.) and is configured to transmit data from, and receive data for, the user device 1310.

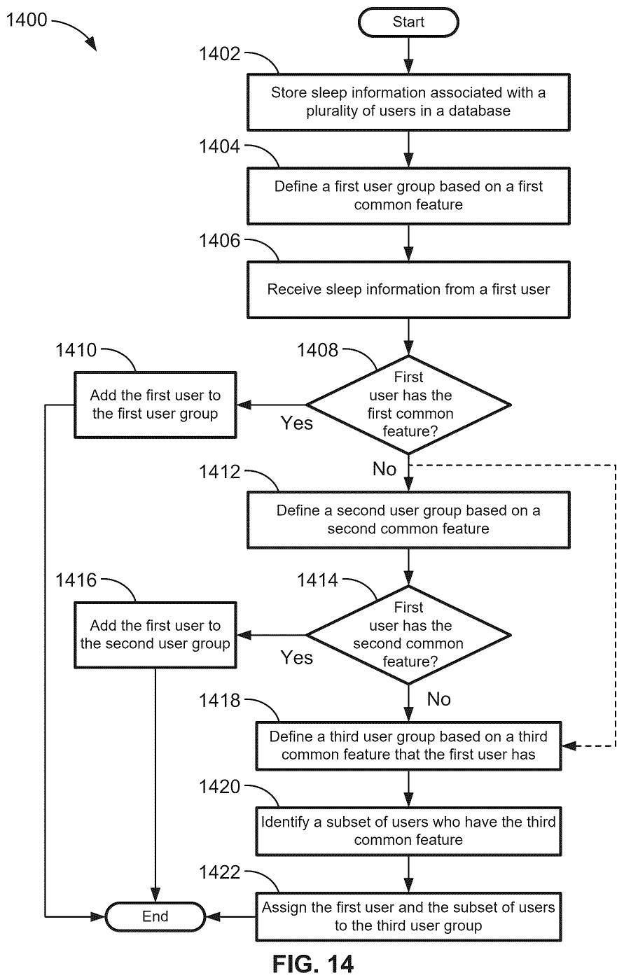

FIG. 14 is a flowchart depicting example operations for assigning users to groups based on sleep information and/or personal information of the user. The flow begins at block 1402.

At block 1402, sleep information associated with a plurality of users is stored. For example, a database can store the sleep information. In some embodiments, at least some of the plurality of users share a first common feature with each other. For example, the first common feature may include any type of sleep information, which can include a sleep diagnosis (e.g., of insomnia, restless leg syndrome, parasomnia, narcolepsy, circadian rhythm sleep-wake disorders, sleep apnea, etc.), a sleep schedule, a duration of sleep, restlessness, severity of sleep disorder, user device usage, use of a type of sleep enhancement device (e.g., sleep enhancement headband), etc. In some embodiments, the sleep information can also include respiratory information. The respiratory information can include any suitable categories. For example, the respiratory information can include use of a respiratory therapy system, a type of respiratory therapy device, a duration of respiratory therapy usage (e.g., how many hours per night a user uses their respiratory therapy system), a length of respiratory therapy usage (e.g., for what period of time, such as days, months, and/or years, a user has been using their respiratory therapy system), a sleep schedule, a duration of sleep, medical conditions, an apnea hypopnea index, a sleep score, etc. According to some embodiments, the respiratory information includes data measured by a respiratory therapy system (e.g., a positive air-pressure machine, a wearable monitoring device, a nearable device, an airable device, or a combination thereof, etc.). According to some embodiments, the database also stores personal information for the plurality of users. The personal information can include any suitable categories. For example, the personal information can include age, gender identity, sex, sexual orientation, location, interests, hobbies, likes, dislikes, preferences, lifestyle choices (e.g., smoker, alcohol use, dietary), etc. The flow continues at block 1404.

At block 1404, a first user group is defined based on the first common feature. For example, a control system can define the first user group based on the first common feature. The first user group can include, for example, a first subset of users selected from the plurality of users. The first subset of users each have the first common feature. According to some embodiments, having the first common feature also includes users who have indicated interest in the first common feature. For example, a user having the first common feature could be a person who is on respiratory therapy or someone who has indicated an interest learning more about respiratory therapy. The first common feature can be checked by the control system based on the sleep information of the plurality of users that has been stored in the database. In some embodiments, some or all of the first subset of users may be assigned to the first user group by the control system automatically. In some embodiments, some or all of the first subset of users may be sent a notification to their respective user devices with an invitation for them to join the first user group. The flow continues at block 1406.

At block 1406, sleep information from a first user is received. For example, a control circuit can receive the sleep information associated with the first user. The control circuit can receive the sleep information from any suitable source, such as a user device, the database, etc. In some embodiments, the control circuit also receives personal information associated with the first user. The flow continues at block 1408.

At block 1408, whether the first user has the first common feature is determined. For example, the control system determines whether the first user has the first common feature. In one embodiment, the control system determines whether the first user has the first common feature by comparing the sleep information and/or the personal information of the first user with the first common feature to determine whether the first user has the first common feature. According to some embodiments, if the control system determines that the first user has the first common feature, the flow continues to block 1410. Otherwise, the control system continues to block 1412. According to some other embodiments, if the control system determines that the first user does not have the first common feature, the flow continues to block 1418 instead of block 1412, as indicated by a third line leading from block 1408 to block 1418.

At block 1410, responsive to the first user having the first common feature, the first user is added. For example, responsive to a control system determining that the first user has the first common feature, the control system adds the first user to the first user group. According to some embodiments, if the control system determines that the first user has the first common feature, the control system may automatically add the first user to the first user group. In some other embodiments, the control system may cause a notification to be transmitted to a user device of the first user to recommend the first user group. If the first user chooses to join the first user group, the control system will then assign the first user as a member of the first user group.

In the event that the control system determines that the first user does not have the first common feature at block 1408, the control system will check whether the first user has a second common feature that is associated with a second user group. It should be noted that the second common feature is only presented as an example. The control system may check the first user for common features with an undefined number of user groups in the attempt to place the first user in a user group.

At block 1412, a second user group is defined. For example, a control system defines the second user group based on a second common feature. According to some embodiments, the second user group could include a second subset of users selected from the plurality of users. In one embodiment, the first user group and the second user group are not mutually exclusive. For example, a user may simultaneous have membership in a plurality of user groups. Similar to the first common feature, according to some embodiments, the second user group may include users who have the second common feature or indicated an interest in the second common feature. According to some embodiments, some or all of the second subset of users may be assigned to the second user group by the control system automatically. In some embodiments, some or all of the second subset of users may be sent a notification to their respective user devices with an invitation for them to join the second user group. The flow continues at block 1414.

At block 1414, whether the first user has the second common feature is determined. For example, responsive to a control system determining that the first user does not have the first common feature, the control system determines whether the first user has the second common feature. According to some embodiments, the control system can compare the sleep information and/or the personal information of the first user to the second common feature to determine whether the first user has the second common feature. If the control system determines that the first user has the second common feature, the flow continues to block 1416. Otherwise, the control system may continue to block 1418.

At block 1416, responsive to determining that the first user has the second common feature, the first user is added to the second user group. For example, responsive to a control system determining that the first user has the second common feature, the control system adds the first user to the second user group. Similar to the first user group, the control system may add the first user to the second user group automatically or cause a notification to be transmitted to a user device of the first user to recommend the second user group.

At block 1418, a third user group is defined based on a third common feature that the first user has. For example, responsive to a control system determining that the first user does not have the first common feature, the control system defines a third user group based on the third common feature. In some embodiments, if the control system cannot find a preexisting user group that has a common feature with the first user, the control system defines a new user group (e.g., the third user group) based on a feature selected from the first user's sleep information and/or personal information (e.g., the third common feature). The control system, for example, may automatically create a third user group based on a third common feature. For example, the third common feature may include a sleep-related-equipment or component thereof (e.g., mask type or type of a sleep enhancement device). The flow continues to block 1420.

At block 1420, a subset of users who have the third common feature in their sleep information are identified. For example, a control system identifies a subset of users who have the third common feature in their sleep information. According to some embodiments, the control system may query the database to return a list of users (e.g., a third subset of users) from the plurality of users who have the third common feature in their sleep information and/or personal information. The flow continues to block 1422.

At block 1422, the first user and the third subset of users are assigned to the third user group. For example, a control system assigns the first user and the third subset of users to the third user group. According to some embodiments, the control system may automatically add the third subset of users and the first user to the third user group. According to some other embodiments, the control system may prompt the first user and/or the third subset of users to join the newly defined group (i.e., the third user group). For example, the control system may cause a recommendation notification to be transmitted to the user devices of the first user and/or the third subset of users with an invitation to join the third user group voluntarily.

The matching application may be a standalone application of a user device. Alternatively, the matching application may be an “opt-in” addition to a pre-existing engagement application that accompanies the user device or external device (e.g., sleep enhancement device, respiratory therapy device, sleep monitoring device, etc.).

One or more elements or aspects or steps, or any portion(s) thereof, from one or more of any of claims 1 to 34 below can be combined with one or more elements or aspects or steps, or any portion(s) thereof, from one or more of any of the other claims 35 to 69 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method comprising:
storing, in a database, sleep information associated with a plurality of users, the plurality of users including a first subset of users having a first common feature in the sleep information;
defining, by a control system based on the first common feature, a first user group comprising the first subset of users;
receiving, via the control system from a user device, sleep information associated with a first user;
determining, by the control system based on the sleep information associated with the first user, whether the first user has the first common feature; and
responsive to determining that the first user has the first common feature, assigning, by the control system, the first user as a constituent of the first user group.

2. The method of claim 1, wherein the plurality of users includes a second subset of users having a second common feature in the sleep information, the method further comprising:
defining, by the control system based on the second common feature, a second user group comprising the second subset of users;
responsive to determining that the first user does not have the first common feature, determining, by the control system based on the sleep information associated with the first user, whether the first user has the second common feature; and
responsive to determining that the first user has the second common feature, assigning, by the control system, the first user as a constituent of the second user group.

3. (canceled)

4. The method of claim 1, wherein the sleep information associated with the plurality of users includes one or more of a sleep diagnosis, a sleep schedule, a duration of sleep, a severity of sleep disorder, restlessness, user device usage, use of a type of sleep enhancement device, and respiratory information.

5. (canceled)

6. The method of claim 4, wherein the respiratory information includes one or more of use of a respiratory therapy system, a type of respiratory therapy device, a duration of respiratory therapy usage, a length of respiratory therapy usage, a sleep schedule, a duration of sleep, medical conditions, an apnea hypopnea index, and sleep score.

7. The method of claim 6, wherein the respiratory information associated with the first user includes data measured by a respiratory therapy system.

8.-9. (canceled)

10. The method of claim 1, further comprising:
storing, in the database, personal information associated with the plurality of users, the plurality of users including a fourth subset of users having a fourth common feature in the personal information;
receiving, via the control system from the first user device, personal information associated with the first user;
receiving, via the control system from the first user device, an instruction to customize user group based on the fourth common feature; and
updating, by the control system, the first user group to comprise a fourth subset of users of the plurality of users having the first common feature and the fourth common feature.

11.-24. (canceled)

25. The method of claim 1, further comprising:
generating, by the control system, a user interface, wherein the user interface includes a group home page associated with the first user group; and
causing, by the control system, presentation of the group home page associated with the first user group on a user device associated with the first user,
wherein the group home page associated with a user group includes one or more of a list of users in the first subset of users, information associated with the first common feature, challenges for user in the subset of users, and a portal to a chatroom.

26. (canceled)

27. The method of claim 1, further comprising:
generating, by the control system, a user interface, wherein the user interface includes a user home page associated with the first user; and
causing, by the control system, presentation of the user home page associated with the first user on a user device associated with the first user,
wherein the home page associated with the user group includes one or more of a list of users in the first subset of users, information associated with the first common feature, challenges for user in the subset of users, a portal to a chatroom, sleep information associated with the first user, and personal information associated the first user.

28. (canceled)

29. The method of claim 1, further comprising:
generating, by the control system based on the sleep information associated with the first user, a recommendation notification; and
causing transmission, by the control system, of the recommendation notification to one or more user device associated with the first user.

30. The method of claim 29, wherein the recommendation notification includes (i) an identification of a second user, the second user being a constituent of the first user group, (ii) an identification of one or more media contents relating to the sleep information associated with the first user or the second user, (iii) a link to a discussion board or a chatroom accessibly to a constituent of the first user group, or (iv) any combination of (i)-(iii).

31.-35. (canceled)

36. A system comprising:
a database, wherein the database is configured to store sleep information associated with a plurality of users; and
a control system communicatively coupled to the database, wherein the control system is configured to:
define, based on a first common feature, a first user group comprising a first subset of users of the plurality of users, the first subset of users having the first common feature in the sleep information;
receive, from a first user device, sleep information associated with a first user;
determine, based on the sleep information associated with the first user, whether the first user has the first common feature; and
responsive to determining that the first user has the first common feature, assigning the first user as constituent of the first user group.

37. The system of claim 36, wherein the plurality of users includes a second subset of users having a second common feature in the sleep information, wherein the control system is further configured to:
define, based on the second common feature, a second user group comprising the second subset of users;
responsive to determining that the first user does not have the first common feature, determine, based on the sleep information associated with the first user, whether the first user has the second common feature; and
responsive to determining that the first user has the second common feature, assigning the first user as a constituent of the second user group.

38. (canceled)

39. The method of claim 35, wherein the sleep information associated with the plurality of users includes one or more of a sleep diagnosis, a sleep schedule, a duration of sleep, severity of sleep disorder, restlessness, user device usage, and use of a type of sleep enhancement device.

40. (canceled)

41. The system of claim 39, wherein the respiratory information includes one or more of use of a respiratory therapy system, a type of respiratory therapy device, a duration of respiratory therapy usage, a length of respiratory therapy usage, a sleep schedule, a duration of sleep, medical conditions, an apnea hypopnea index, and sleep score.

42. The system of claim 41, wherein the sleep information associated with the first user includes data measured by a respiratory therapy system.

43.-44. (canceled)

45. The system of claim 35, wherein:
the database is further configured to store personal information associated with the plurality of users, the plurality of users including a fourth subset of users having a fourth common feature in the personal information; and
the control system is further configured to:
receive from the first user device, personal information associated with the first user;
receive from the first user device, an instruction to customize user group based on the fourth common feature; and
update the user group to comprise a fourth subset of users of the plurality of users having the first common feature and the fourth common feature.

46.-59. (canceled)

60. The system of claim 35, wherein the control system is further configured to:
generate a user interface, wherein the user interface includes a group home page associated with the first user group; and
cause presentation of the group home page associated with the first user group on a user device associated with the first user,
wherein the group home page associated with a user group includes one or more of a list of users in the first subset of users, information associated with the first common feature, challenges for user in the subset of users, and a portal to a chatroom.

61. (canceled)

62. The system of claim 35, wherein the control system is further configured to:
generate a user interface, wherein the user interface includes a user home page associated with the first user; and
cause presentation of the user home page associated with the first user on a user device associated with the first user,
wherein the home page associated with the user group includes one or more of a list of users in the first subset of users, information associated with the first common feature, challenges for user in the subset of users, a portal to a chatroom, sleep information associated with the first user, and personal information associated the first user.

63. (canceled)

64. The system of claim 35, wherein the control system is further configured to:
generate, based on the sleep information associated with the first user, a recommendation notification; and
cause transmission of the recommendation notification to one or more user device associated with the first user.

65. The system of claim 64, wherein the recommendation notification includes (i) an identification of a second user, the second user being a constituent of the first user group, (ii) an identification of one or more media contents relating to the sleep information associated with the first user or the second user, (iii) a link to a discussion board or a chatroom accessibly to a constituent of the first user group, or (iv) any combination of (i)-(iii).

66.-69. (canceled)
